(19) United States
(12) Patent Application Publication
(10) Pub. No.: US 2025/0041414 A1
(43) Pub. Date: Feb. 6, 2025

(54) DENDRITIC CELL CANCER VACCINE AND APPLICATION THEREOF

(71) Applicant: SUZHOU ERSHENG BIOMEDICAL CO., LTD., Suzhou, Jiangsu (CN)

(72) Inventors: Mi LIU, Suzhou (CN); Lu DIAO, Suzhou (CN)

(73) Assignee: SUZHOU ERSHENG BIOMEDICAL CO., LTD., Suzhou, Jiangsu (CN)

(21) Appl. No.: 18/718,623

(22) PCT Filed: Jan. 21, 2022
(86) PCT No.: PCT/CN2022/073141

§ 371 Date: Jun. 11, 2024

(30) Foreign Application Priority Data
Dec. 24, 2021 (CN) 202111603486.4

(51) Int. Cl. A61K 39/00; A61P 35/00

(52) U.S. Cl. CPC A61K 39/4615; A61K 39/0011; A61P 35/00; A61K 2039/5154; A61K 2039/5158; A61K 2039/55555

(57) ABSTRACT

A dendritic cell cancer vaccine obtained by activating dendritic cells in vitro with delivery particles loaded with cell components is provided, wherein the delivery particles are nanoparticles and/or micronparticles, the cell components are derived from water-soluble components and/or non-water-soluble components of cancer cells and/or tumor tissues, the activating is co-incubating the delivery particles loaded with the cell components with the dendritic cells.

### TECHNICAL FIELD

The present disclosure relates to the field of immunotherapy technology, and in particular, relates to a dendritic cell cancer vaccine and application thereof.

### BACKGROUND

Immunity is a physiological function of the human body, which relies on this function to recognize “self” and “non-self” components, thereby destroying and clearing abnormal substances (such as viruses, bacteria, etc.) in the human body, or damaged cells and tumor cells produced by the human body itself, to maintain human health. In recent years, immune technology has developed rapidly, especially in the field of cancer immunotherapy. With the continuous improvement of understanding of cancer, people have found that the human immune system and various immune cells play a key role in inhibiting the occurrence and development of cancer. By regulating the balance of the body's immune system, we have the potential to influence and control the occurrence, development, and treatment of cancer.

Cancer vaccines are one of the important methods in cancer immunotherapy and prevention. The dendritic cell (DC) is the most important antigen-presenting cell and the main cell activated by characteristic specific immune reactions. Dendritic cells originate from bone marrow lymphocytes and can settle in tissues throughout the body, monitor the surrounding environment, and transmit captured information to the adaptive immune system (T lymphocytes (hereinafter referred to as T cells) and B lymphocytes (hereinafter referred to as B cells)) at any time. They are specialized antigen-presenting cells that express major histocompatibility complex (MHC) class I and II molecules, and are key links between innate and adaptive immunity. Dendritic cells internalize and decompose antigens obtained from the periphery into short peptide segments, which are expressed on the surface of dendritic cells in the form of peptide-MHC complexes. This process is also the process of dendritic cell maturation, and then dendritic cells carrying antigen peptides migrate to the secondary lymphoid organs, where T cells are activated. Compared with other antigen-presenting cells, dendritic cells have extremely high antigen-presenting efficiency and can induce a handful of T cell responses, becoming the most effective endogenous stimulus for T and B cell responses.

Dendritic cell cancer vaccine is a type of cancer vaccine. Currently, the development of dendritic cancer vaccines mainly involves selecting several peptide antigens or protein antigens or the supernatant of cancer cell lysate to activate dendritic cells in vitro, and then infusing them back into the body. At present, research has shown that dendritic cells around tumors can capture tumor antigens released by tumor cells. These antigens come from dead tumor cells or live tumor cells that are phagocytosed by dendritic cells. These antigens are then cross-presented to tumors to drain T cells from lymph nodes, thereby inducing the production of tumor antigen-specific cytotoxic T lymphocytes (CTLs) and killing tumor cells. However, in clinical practice, due to the overall immunosuppressive environment of tumors and the multiple cell interactions in the local immune microenvironment of tumors, dendritic cells are unable to complete immune responses successfully. Moreover, in the tumor microenvironment, tumor cells express and secrete multiple molecules that can inhibit dendritic cell activation and drive dendritic cells to transform into inhibitory or regulatory phenotypes, inhibiting the immune response of tumors. Therefore, even though dendritic cell vaccines have certain effects, not all clinical trials have shown that dendritic cell vaccines can benefit the survival of cancer patients. For example, Walker et al.'s Phase I clinical trial included 9 patients with glioblastoma and 4 patients with anaplastic astrocytoma. Dendritic cell vaccines combined with standard glioma treatment were administered. After reoperation, T cell infiltration was increased in tumor samples, but the overall survival period was not significantly prolonged. In 2010, the U.S. Food and Drug Administration (FDA) approved the first dendritic cell vaccine Provenge for the treatment of refractory prostate cancer. However, since only several limited antigens were used in the preparation process to stimulate and activate dendritic cells in vitro, the clinical efficacy of the Provenge vaccine is limited. Since then, the dendritic cell vaccine has achieved certain efficacy in the treatment of breast cancer, bladder cancer, kidney cancer, colon and rectal cancer, lung cancer, and melanoma, but it only activates dendritic cells through partial antigens, and the therapeutic effect still needs to be improved.

The basis of cancer vaccines is to select appropriate cancer antigens to activate the human immune system's recognition of abnormally mutated cancer cells. Cancer cells and tumor tissues have high heterogeneity and numerous mutations, so cancer cells or tumor tissues themselves are the best sources of cancer antigens. The more dendritic cells there are, the more antigens they phagocytose, and the better the efficacy of the vaccine. However, directly mixing and incubating tumor lysate with dendritic cells is not easy for dendritic cells to phagocytose and uptake, as the cell membrane is lipophilic and the components in the supernatant of the cell lysate previously used by researchers are water-soluble. Therefore, a new method for preparing dendritic cell vaccines is still needed.

### SUMMARY

In order to solve the aforementioned technical problems, the present disclosure provides a dendritic cell vaccine based on micronparticles or nanoparticles loaded with whole cell components or a mixture thereof of one or more kinds of cancer cells and/or one or more kinds of tumor tissues to activate dendritic cells in vitro.

The dendritic cell cancer vaccine of the present disclosure is obtained by activating the dendritic cells in vitro with delivery particles loaded with the cell components, wherein the delivery particles are nanoparticles and/or micronparticles, the cell components are derived from water-soluble and/or non-water-soluble components of one or more kinds of the cancer cells and/or cells in one or more kinds of tumor tissues, and the activating refers to co-incubating the delivery particles loaded with the cell components with the dendritic cells.

In the present disclosure, the cell components are derived from the components obtained from one or more kinds of the cancer cells and/or whole cells of one or more kinds of the tumor tissue. Loading the non-water-soluble components onto the delivery particles increased antigens in the vaccine system. More preferably, both the water-soluble and the non-water-soluble components are loaded onto the delivery particles together, so that all antigens are loaded onto the delivery particles, which are then co-incubated with the dendritic cells in vitro. After being phagocytosed by the dendritic cells, the delivery particles may be phagocytosed by the dendritic cells for the antigen presentation and activation. After infusing back into the body, the delivery particles may home lymph nodes and activate tumor-specific T cells by the antigens loaded on DC cells.

Furthermore, the dendritic cells are autologous and/or allogeneic dendritic cells. The present disclosure uses in vitro activation of the dendritic cells instead of in vivo activation because the inventor has found through extensive experiments that due to the different environments of in vivo and in vitro activation, the presentation function of DC cells may not be fully utilized in vivo, resulting in differences in therapeutic efficacy.

Furthermore, the dendritic cells originate from any cells that can be used to prepare and isolate the dendritic cells, including but not limited to stem cells, bone marrow cells, and peripheral immune cells.

Furthermore, the delivery particles and loaded the cellular components thereof are co-incubated with the dendritic cells for at least 4 hours, allowing the cellular components loaded on micron/nanoparticles to be delivered into the dendritic cells and processed and antigen-presenting by the dendritic cells. In the embodiments described below, the co-incubation time is at least 4 hours, preferably 48-96 hours.

Furthermore, the water-soluble components in the present disclosure are the original water-soluble parts in cells or tissues that is soluble in pure water or aqueous solutions without solubilizer, while the non-water-soluble components are the original non-water-soluble parts of cells or tissues that change from insoluble in pure water to soluble in aqueous solutions with solubilizer or organic solvents using appropriate solubilization methods. Wherein the cellular components are obtained by lysing whole cells of one or more kinds of cancer cells and/or tumor tissues, or by processing the lysed whole cells of one or more kinds of cancer cells and/or tumor tissues, or by lysing the processed whole cell of one or more kinds of cancer cells and/or tumor tissues.

Furthermore, both the water-soluble and non-water-soluble parts of the cell components can be dissolved by solubilized aqueous solutions or organic solvents with solubilizer. The solubilizer is at least one of the solubilizers that can increase the solubility of proteins or polypeptides in aqueous solutions: the organic solvents are organic solvents that can dissolve proteins or polypeptides.

Furthermore, solubilizers include but are not limited to urea, guanidine hydrochloride, sodium deoxycholate, SDS, glycerol, alkaline solution with pH greater than 7, acidic solution with pH less than 7, various protein degrading enzyme, albumin, lecithin, high concentration inorganic salt, Triton, Tween, DMSO, acetonitrile, ethanol, methanol, DMF, propanol, isopropanol, acetic acid, cholesterol, amino acid, glycoside, choline, Brij™-35, octaethylene glycol monododecyl ether, CHAPS, Digitonin, lauryldimethylamine oxide, IGEPAL® CA-630. Those skilled in the art can understand that the non-water-soluble components may also be changed from insoluble in pure water to soluble by other methods that can increase the solubility of proteins and polypeptide fragments. Organic solvents include but are not limited to DMSO, acetonitrile, ethanol, methanol, DMF, isopropanol, propanol, dichloromethane, and ethyl acetate. Those skilled in the art can understand that the organic solvents may also use other methods containing organic solvents that can solubilize proteins and polypeptide fragments.

Furthermore, the nanoparticles and/or micronparticles system for activating the dendritic cells includes particles with nanometer size or micrometer size and the cell components or the mixtures thereof loaded on the particles. The mixtures include but are not limited to mixing water-soluble components, or mixing non-water-soluble components, or mixing all or part of water-soluble components with all or part of non-water-soluble components.

Furthermore, the cell components or the mixtures thereof are loaded inside and/or on surfaces of micron/nanoparticles. Specifically, the loading method is that the water-soluble and non-water-soluble components of the cells are respectively or together loaded inside the particles, and/or respectively or together loaded on the surfaces of the particles, including but not limited to the water-soluble components are together loaded inside and on the surfaces of the particles, the non-water-soluble components are together loaded inside and on the surfaces of the particles, the water-soluble components are loaded inside the particles while the non-water-soluble components are loaded on the surfaces of the particles, the non-water-soluble components are loaded inside the particles while the water-soluble components are loaded on the surfaces of the particles, the water-soluble and the non-water-soluble components are loaded inside the particles while only the non-water-soluble components are loaded on the surfaces of the particles, the water-soluble and the non-water-soluble components are loaded inside the particles while only the water-soluble components are loaded on the surfaces of the particles, the water-soluble components are loaded insides the particles while both the water-soluble and the non-water-soluble components are together loaded on the surfaces of the particles, the non-water-soluble components are loaded inside the particles while both the water-soluble and the non-water-soluble components are together loaded on the surfaces of the particles, and both the water-soluble and the non-water-soluble components are together loaded inside the particles while both the water-soluble and the non-water-soluble components are together loaded on the surfaces of the particles.

Furthermore, immunopotentiating adjuvants are loaded inside the delivery particles, and/or the immunopotentiating adjuvants are loaded on the surfaces of the delivery particles. The immunopotentiating adjuvants include but are not limited to at least one of immunopotentiators derived from microorganisms, products of human or animal immune systems, innate immune agonists, adaptive immune agonists, chemical synthetic drugs, fungal polysaccharides, traditional Chinese medicine, and other categories: the immunopotentiating adjuvants include but are not limited to at least one of pattern recognition receptor agonists, Bacillus Calmette-Guérin vaccine (BCG), manganese related adjuvants, cell-wall skeleton of BCG, methanol extraction residue of BCG, muramyl dipeptide of BCG, mycobacterium phleis, polyactin A, mineral oil, virus-like particles, immunopotentiating reconstituted influenza virosomes, cholera enterotoxin, saponins and derivatives thereof, resiquimod, thymosin, liver active peptide of newborn bovine, imiquimod, polysaccharides, curcumin, immuno adjuvant CpG, immuno adjuvant poly(I:C), immuno adjuvant poly ICLC, Corynebacterium Parvum vaccine, hemolytic streptococcus preparation, coenzyme Q10, levamisole, polycytidylic acid, manganese adjuvants, aluminum adjuvants, calcium adjuvants, various cytokines, interleukin, interferon, polyinosinic acid, polyadenosinic acid, alum, aluminum phosphate, lanolin, squalene, cytokines, vegetable oil, endotoxin, liposome adjuvants, MF59, double-stranded RNA, double-stranded DNA, aluminum related adjuvants, CAF01, and effective ingredients from ginseng or Astragalus Membranaceus. Those skilled in the art can understand that the list herein is not exhaustive, and the immunopotentiating adjuvants may also use other substances that can enhance the immune response.

Furthermore, co-loading immunopotentiating adjuvants with the cell components inside nanoparticles or micronparticles can better activate the dendritic cells after nanoparticles or micronparticles are phagocytosed by the dendritic cells, facilitating their ability to prevent or treat cancer after infusing back into the body.

Furthermore, the surfaces of the micron/nanoparticles may not be connected to target heads with active targeting function, or connected to the target heads with active targeting function. The target heads may be commonly used target heads such as mannose, CD32 antibody, CD11c antibody, CD103 antibody, CD44 antibody, etc., leading the particle system to target and deliver into the dendritic cells.

Furthermore, the micron/nanoparticle systems loaded with the cell components may be prepared by existing preparation methods, including but not limited to common solvent evaporation, dialysis, extrusion, and hot melt process. In some embodiments, it is prepared by the double emulsion method in solvent evaporation.

Furthermore, the nanoparticles and/or the micronparticles (nanoparticles and/or micronparticles referred to as micron/nanoparticles in the present disclosure) may not be modified during the preparation process, or appropriate modification techniques can be used to increase the antigen loading capacity and/or immunogenicity of nanovaccine or microvaccine to enhance the therapeutic effect of the dendritic cell vaccine.

Furthermore, the form in which the cell components or the mixtures thereof are loaded inside nano and/or micronparticles is any way in which cell components or the mixtures thereof may be loaded inside nano and/or micronparticles.

Furthermore, the methods in which cell components or the mixtures thereof are loaded onto the surfaces of the nanoparticles and/or the micronparticles include but are not limited to adsorption, covalent bonding, charge interactions (such as adding positively charged substances, adding negatively charged substances), hydrophobic interactions, one-step or multi-step solidification, mineralization, encapsulation, etc.

Furthermore, the water-soluble components and/or the non-water-soluble components loaded on the surfaces of the micron/nanoparticles are one or more layers after loaded, and when the surface of vaccine are loaded with multiple layers of the water-soluble components and/or the non-water-soluble components, there are modifiers between the layers.

Furthermore, the particle size of nanoparticles or micronparticles is nanoscale or micronscale, which can ensure that the vaccine is phagocytosed by antigen-presenting cells. To improve phagocytic efficiency, the particle size should be within an appropriate range. The particle size of the nanoparticles is 1 nm-1000 nm, more preferably, the particle size is 30 nm-1000 nm, and most preferably, the particle size is 100 nm-600 nm. The particle size of micronparticles is 1 μm-1000 μm, more preferably, the particle size is 1 μm-10 μm, more preferably, the particle size is 1 μm-10 μm, and most preferably, the particle size is 1 μm-5 μm.

Furthermore, the surface of the micron/nanoparticles may be electric neutral, negatively charged, or positively charged.

Furthermore, preparation materials for the micron/nanoparticles are organic synthetic polymer materials, natural polymer materials, or inorganic materials. Among them, the organic synthetic polymer materials are biocompatible or degradable polymer materials, including but not limited to poly(lactic-co-glycolic acid) copolymers PLGA, PLA, PGA, Poloxamer, PEG, PCL, PEI, PVA, PVP, PTMC, polyanhydride, PDON, PPDO, PMMA, poly(amino acids), synthetic peptides, and synthetic lipids. The natural polymer materials are biocompatible or degradable polymer materials, including but not limited to lecithins, cholesterols, starch, lipids, sugars, peptides, sodium alginates, albumins, collagens, gelatins, and cell membrane components. The inorganic materials are materials without obvious biological toxicity, including but not limited to ferric oxide, ferroferric oxide, calcium carbonate, calcium phosphate, etc.

Furthermore, the shapes of micron/nanoparticles are any common shapes, including but not limited to spherical, spheroidal, dolioform, polygonal, rod-shaped, sheet-shaped, linear, worm-shaped, square, triangular, butterfly-shaped, or disc-shaped.

In some embodiments, a specific preparation method of the double emulsion method in the present disclosure is as follows:

Step 1, adding the first predetermined volume of aqueous solution with the first predetermined concentration to the second predetermined volume of the organic phase with the second predetermined concentration of medical polymer material.

In some embodiments, the aqueous solution may contain various components of cancer cell lysates, as well as immunopotentiating adjuvants poly(I:C), BCG, manganese adjuvants, calcium adjuvants, or CpG. The components in the cancer cell lysate are either water-soluble or original no-water-soluble components that solubilized in urea or guanidine hydrochloride during preparation. In the aqueous solution, the concentration of the water-soluble components derived from the cancer cells or the concentration of the original non-water-soluble components derived from the cancer cells that solubilized in urea or guanidine hydrochloride, i.e. the first predetermined concentration requires the concentration of protein/peptides greater than 1 ng/ml to load sufficient cancer antigens to activate relevant immune responses. The concentration of the immunopotentiating adjuvants in the initial aqueous phase is greater than 0.01 ng/ml.

In some embodiments, the aqueous solution contains various components of tumor tissue lysates, as well as immunopotentiating adjuvants poly(I:C), BCG, manganese adjuvants, calcium adjuvants, or CpG. The components in the tumor tissue lysate are either water-soluble or original no-water-soluble components that solubilized in the urea or guanidine hydrochloride during preparation. In the aqueous solution, the concentration of the water-soluble components derived from the tumor tissues or the concentration of the original non-water-soluble components derived from the tumor tissues and dissolved in the urea or guanidine hydrochloride, i.e. the first predetermined concentration requires the concentration of protein/peptides greater than 0.01 ng/ml to load sufficient cancer antigens to activate relevant immune responses. The concentration of the immunopotentiating adjuvants in the initial aqueous phase is greater than 0.01 ng/ml.

In the present disclosure, medical polymer materials are dissolved in the organic solvents to obtain the second predetermined volume of the organic phase with the second predetermined concentration of the medical polymer materials. In some examples, the medical polymer materials are PLGA, and the organic solvents are dichloromethane. In addition, in some examples, the range of the second predetermined concentration of the medical polymer materials is 0.5 mg/mL to 5000 mg/mL, preferably 100 mg/mL.

In the present disclosure, PLGA or modified PLGA is chosen because it is a biodegradable material and has been approved by the FDA as a medical dressing. Research has shown that PLGA has certain immune regulatory functions, which makes it suitable as an excipient for the preparation of the nanoparticles or the micronparticles.

In practice, the second predetermined volume of the organic phase is set based on its ratio to the first predetermined volume of the aqueous phase. In the present disclosure, the ratio of the first predetermined volume of the aqueous phase to the second predetermined volume of the organic phase ranges from 1:1.1 to 1:5000, preferably 1:10. In a specific implementation process, the first predetermined volume, the second predetermined volume, and the ratio of the first predetermined volume to the second predetermined volume may be adjusted as needed to adjust the size of the prepared nanoparticles or micronparticles.

Preferably, when the aqueous solution is a solution of lysate components, wherein the concentration of the proteins and peptides is greater than 1 ng/mL, preferably 1 mg/mL to 100 mg/mL. When the aqueous solution is the component solution of the lysate components/immune adjuvants, the concentration of the proteins and peptides is greater than 1 ng/ml, preferably 1 mg/mL to 100 mg/mL, and the concentration of the immune adjuvants is greater than 0.01 ng/ml, preferably 0.01 mg/mL to 20 mg/mL. In a organic phase solution of the polymer materials, solvents are DMSO, acetonitrile, ethanol, chloroform, methanol, DMF, isopropanol, dichloromethane, propanol, ethyl acetate, etc., preferably dichloromethane. The concentration of the polymer materials is 0.5 mg/mL to 5000 mg/mL, preferably 100 mg/mL. A first emulsifier solution is preferably a polyvinyl alcohol aqueous solution with a concentration of 10 mg/mL to 50 mg/mL, preferably 20 mg/mL. A second emulsifier solution is preferably a polyvinyl alcohol aqueous solution with a concentration of 1 mg/mL to 20 mg/mL, preferably 5 mg/mL. A dispersion solution is PBS buffer or normal saline or pure water.

Step 2, performing ultrasonic treatment for more than 2 seconds, or stirring for more than 1 minute or homogenization treatment, or microfluidic treatment on the mixture obtained from Step 1. Preferably, when the stirring is mechanical or magnetic stirring, the stirring speed should be greater than 50 rpm and the stirring time should be greater than 1 minute. For example, the stirring speed is between 50 rpm to 1500 rpm, the stirring time is between 0.1 hours to 24 hours. During the ultrasonic treatment, the ultrasonic power is greater than 5 W and the time is greater than 0.1 seconds, such as 2 to 200 seconds. During the homogenization treatment, use a high-pressure/ultra-high-pressure homogenizer or a high-shear homogenizer. When using the high-pressure/ultra-high-pressure homogenizer, the pressure is greater than 5 psi, such as 20 psi to 100 psi. When using the high-shear homogenizer, the speed is greater than 100 rpm, such as 1000 rpm to 5000 rpm. When using the microfluidic treatment, the flow rate is greater than 0.01 mL/min, such as 0.1 mL/min to 100 mL/min. Nanoization and/or micronization may be achieved through ultrasound, stirring, homogenization treatment, or microfluidic treatment. Ultrasound duration, or stirring speed, or homogenization pressure and time can control the size of the prepared micro/nanoparticles, it will cause changes in particle size if it is too large or too small.

Step 3, adding the mixture obtained from step 2 to the third predetermined volume of an aqueous solution containing the third predetermined concentration of emulsifier and perform ultrasonic treatment for more than 2 seconds, or stirring for more than 1 minute, or perform homogenization treatment or microfluidic treatment. This step adds the mixture obtained from step 2 to the emulsifier aqueous solution and continues to ultrasonic or stir for nanoization or micronization. This step is to achieve nanoization or micronization, and the length of ultrasonic time, or stirring speed and time can control the size of the prepared nanoparticles or micronparticles. It will cause changes in particle size if it is too long or too short, therefore, it is necessary to choose an appropriate ultrasound time. In the present disclosure, the ultrasonic time is greater than 0.1 seconds, such as 2 to 200 seconds; the stirring speed is greater than 50 rpm, such as 50 to 500 rpm; and the stirring time is greater than 1 minute, such as 60 to 6000 seconds. Preferably, when the stirring is mechanical or magnetic stirring, the stirring speed is greater than 50 rpm and the stirring time is greater than 1 minute. For example, the stirring speed is between 50 rpm to 1500 rpm, and the stirring time is between 0.5 hours to 5 hours. During the ultrasonic treatment, the ultrasonic power is between 50 W to 500 W, and the time is greater than 0.1 seconds, such as 2 to 200 seconds. During the homogenization treatment, use a high-pressure/ultra-high-pressure homogenizer or a high-shear homogenizer. When using the high-pressure/ultra-high-pressure homogenizer, the pressure is greater than 20 psi, such as 20 psi to 100 psi. When using the high-shear homogenizer, the speed is greater than 1000 rpm, such as 1000 rpm to 5000 rpm. When using the microfluidic treatment, the flow rate is greater than 0.01 mL/min, such as 0.1 mL/min to 100 mL/min. Nanoization and/or micronization may be achieved through sonication, or stirring, or homogenization treatment, or microfluidic treatment. Ultrasonic duration, or stirring speed, or homogenization pressure and time can control the size of the prepared nano/micronparticles, it will cause changes in particle size if it is too large or too small.

In the present disclosure, the emulsifier aqueous solution is a polyvinyl alcohol (PVA) aqueous solution, with the third predetermined volume of 5 mL and the third predetermined concentration of 20 mg/mL. The third predetermined volume is adjusted according to its ratio to the second predetermined volume. In the present disclosure, a range between the second and the third predetermined volumes is set from 1:1.1 to 1:1000, preferably may be 2:5. In the procedure of the specific embodiment, to control the size of the nanoparticles or the micronparticles, a ratio of the second and the third predetermined volumes may be adjusted. Similarly, a values of ultrasonic time or stirring time, a volume of emulsifier aqueous solution, and a concentration in this step are all based on the purpose of obtaining the nanoparticles or the micronparticles with appropriate size.

Step 4, adding the liquid obtained from step 3 to a fourth predetermined volume emulsifier aqueous solution with a fourth predetermined concentration, and stirring until the predetermined stirring conditions are met.

In this step, the emulsifier aqueous solution is still PVA.

The fourth predetermined concentration is 5 mg/mL, and selection of the fourth predetermined concentration is based on obtaining the nanoparticles or the micronparticles with appropriate size. The selection of the fourth predetermined volume is determined by a ratio of the third predetermined volume to the fourth predetermined volume. In the present disclosure, the ratio of the third predetermined volume to the fourth predetermined volume ranges from 1:1.5 to 1:2000, preferably 1:10. In the procedure of the specific embodiment, to control the size of nanoparticles or micronparticles, the ratio of the third and the fourth predetermined volumes may be adjusted.

In the present disclosure, the predetermined stirring condition for this step is until the organic solvent fully volatilizes, that is, the dichloromethane volatilization in step 1 fully volatilizes.

Step 5, centrifuging the mixture processed in step 4 that meets the predetermined stirring conditions at a speed greater than 100 RPM for more than 1 minute, removing the supernatant, and resuspending the remaining precipitate in the fifth predetermined volume aqueous solution with the fifth predetermined concentration and containing freeze-drying protectant, or in the sixth predetermined volume PBS (or normal saline).

In some embodiments of the present disclosure, freeze-drying is not required when resuspending the residue obtained in step 5 in the sixth predetermined volume PBS (or normal saline), and subsequent experiments related to the adsorption of the cancer cell lysates on the surfaces of the nanoparticles or the micronparticles may be directly carried out.

In some embodiments of the present disclosure, the freeze-drying is required when resuspending the residue obtained in step 5 in an aqueous solution with freeze-drying protectant, experiments related to the adsorption of the cancer cell lysates on the surfaces of the nanoparticles or the micronparticles are conducted after the freeze-drying.

In the present disclosure, the freeze-drying protectant is trehalose.

In the present disclosure, the fifth predetermined concentration of the freeze-drying protectant in this step is 4% by mass. The reason for this setting is not to affect the freeze-drying effect in subsequent freeze-drying.

Step 6, freeze-drying the suspension containing the freeze-drying protectant obtained in step 5, and then reserving freeze-dried substance for later use.

Step 7, mixing the sixth predetermined volume of suspension containing the nanoparticles resuspended in PBS (or normal saline) obtained in step 5 or resuspend the freeze-dried substance containing the nanoparticles or the micronparticles and freeze-dried protectants after the freeze-drying obtained in step 6 by the sixth predetermined volume of PBS (or normal saline), with the seventh predetermined volume of the water-soluble components or the original non-water-soluble components solubilized in 8M urea to obtained the nanoparticle or the micronparticle systems.

In the present disclosure, a volume ratio of the sixth predetermined volume to the seventh predetermined volume is 1:10000 to 10000:1, preferably the volume ratio is 1:100 to 100:1, most preferably the volume ratio is 1:30 to 30:1.

In some embodiments, when the volume of the resuspended nanoparticle suspension is 10 mL, the volume of the cancer cell lysate or the water-soluble components containing the tumor tissue lysates or the original non-water-soluble components solubilized in 8M urea is 1 mL. In actual use, the volume and ratio of these may be adjusted as needed.

Step 8, mixing nanoparticles and/or micronparticles prepared in step 7 with the dendritic cells and incubate for a certain period.

Step 9, collecting activated dendritic cells from step 8 and reintroducing them into the body for cancer prevention or treatment.

In other embodiments, a specific preparation method of the double emulsion method in the present disclosure is as follows:

Step 1, adding the first predetermined volume of aqueous solution with the first predetermined concentration to the second predetermined volume of the organic phase with the second predetermined concentration of the medical polymer materials.

In some embodiments, the aqueous solution may contain various components of the cancer cell lysates, as well as immunopotentiating adjuvants poly(I:C), manganese adjuvants, calcium adjuvants, BCG or CpG. The components in the cancer cell lysates are either water-soluble or original no-water-soluble components dissolved in urea or guanidine hydrochloride during preparation. In the aqueous solution, the concentration of the water-soluble components derived from the cancer cells or the concentration of the original non-water-soluble components derived from the cancer cells and solubilized in the urea or guanidine hydrochloride, i.e. the first predetermined concentration requires the concentration of protein/peptides greater than 0.01 ng/ml to load sufficient cancer antigens to activate relevant immune responses. The concentration of the immunopotentiating adjuvants in the initial aqueous phase is greater than 0.01 ng/ml.

In some embodiments, the aqueous solution contains various components of the tumor tissue lysates, as well as immunopotentiating adjuvants poly(I:C), manganese adjuvants, calcium adjuvants, BCG or CpG. The components in tumor tissue lysate are either water-soluble or original no-water-soluble components dissolved in the urea or guanidine hydrochloride during preparation. In the aqueous solution, the concentration of the water-soluble components derived from the tumor tissues or the concentration of the original non-water-soluble components derived from the tumor tissues and dissolved in the urea or guanidine hydrochloride, i.e. the first predetermined concentration requires the concentration of the protein/peptides greater than 0.01 ng/ml to load sufficient cancer antigens to activate relevant immune responses. The concentration of immunopotentiating adjuvants in the initial aqueous phase is greater than 0.01 ng/ml.

In the present disclosure, the medical polymer materials are dissolved in the organic solvents to obtain the second predetermined volume of the organic phase with the second predetermined concentration of the medical polymer materials. In some examples, the medical polymer materials are PLGA, and the organic solvents are dichloromethane. In addition, in some examples, the range of the second predetermined concentration of medical polymer materials is 0.5 mg/mL to 5000 mg/mL, preferably 100 mg/mL.

In the present disclosure, PLGA or modified PLGA is chosen because it is a biodegradable material and has been approved by the FDA as a medical dressing. Research has shown that PLGA has certain immune regulatory functions, which makes it suitable as an excipient for the preparation of nanoparticles or micronparticles.

In practice, the second predetermined volume of the organic phase is set based on its ratio to the first predetermined volume of the aqueous phase. In the present disclosure, the ratio of the first predetermined volume of the aqueous phase to the second predetermined volume of the organic phase ranges from 1:1.1 to 1:5000, preferably 1:10. In the specific implementation process, the first predetermined volume, the second predetermined volume, and the ratio of the first predetermined volume to the second predetermined volume may be adjusted as needed to adjust the size of the prepared nanoparticles or micronparticles.

Preferably, when the aqueous solution is the solution of the lysate components, wherein the concentration of the proteins and peptides is greater than 1 ng/mL, preferably 1 mg/mL to 100 mg/mL. When the aqueous solution is the solution of the lysate components/immune adjuvants, the concentration of the proteins and peptides is greater than 1 ng/ml, preferably 1 mg/mL to 100 mg/mL, and the concentration of the immune adjuvants is greater than 0.01 ng/ml, preferably 0.01 mg/mL to 20 mg/mL. In the organic phase solution of the polymer materials, the solvents are DMSO, acetonitrile, ethanol, chloroform, methanol, DMF, isopropanol, dichloromethane, propanol, ethyl acetate, etc., preferably dichloromethane. The concentration of the polymer materials is 0.5 mg/mL to 5000 mg/mL, preferably 100 mg/mL. The first emulsifier solution is preferably a polyvinyl alcohol aqueous solution with a concentration of 10 mg/mL to 50 mg/mL, preferably 20 mg/mL. The second emulsifier solution is preferably a polyvinyl alcohol aqueous solution with a concentration of 1 mg/mL to 20 mg/mL, preferably 5 mg/mL. The dispersion solution is PBS buffer or normal saline or pure water.

Step 2, performing sonication treatment for more than 2 seconds, or stirring for more than 1 minute, or homogenization treatment, or microfluidic treatment on the mixture obtained from Step 1. Preferably, when the stirring is mechanical or magnetic stirring, the stirring speed should be greater than 50 rpm and the stirring time should be greater than 1 minute. For example, the stirring speed is between 50 rpm to 1500 rpm, the stirring time is between 0.1 hours to 24 hours. During the ultrasonic treatment, the ultrasonic power is greater than 5 W and the time is greater than 0.1 seconds, such as 2 to 200 seconds. During the homogenization treatment, use a high-pressure/ultra-high-pressure homogenizer or a high-shear homogenizer. When using the high-pressure/ultra-high-pressure homogenizer, the pressure is greater than 5 psi, such as 20 psi to 100 psi. When using the high-shear homogenizer, the speed is greater than 100 rpm, such as 1000 rpm to 5000 rpm. When using microfluidic treatment, the flow rate is greater than 0.01 mL/min, such as 0.1 mL/min to 100 mL/min. Nanoization and/or micronization may be achieved through ultrasound, stirring, homogenization treatment, or microfluidic treatment. Ultrasound duration, or stirring speed, or homogenization pressure and time can control the size of the prepared micro/nanoparticles, it will cause changes in particle size if it is too large or too small.

Step 3, adding the mixture obtained from step 2 to the third predetermined volume of an aqueous solution containing the third predetermined concentration of emulsifier and perform ultrasonic treatment for more than 2 seconds, or stirring for more than 1 minute, or performing homogenization treatment or microfluidic treatment. This step adds the mixture obtained from step 2 to the emulsifier aqueous solution and continues to ultrasonic or stir for nanoization or micronization. This step is to achieve nanoization or micronization, and ultrasonic duration, or stirring speed and time can control the size of the prepared nanoparticles or micronparticles. It will cause changes in particle size if it is too long or too short, therefore, it is necessary to choose an appropriate ultrasound time. In the present disclosure, the ultrasonic time is greater than 0.1 seconds, such as 2 to 200 seconds: the stirring speed is greater than 50 rpm, such as 50 to 500 rpm; and the stirring time is greater than 1 minute, such as 60 to 6000 seconds. Preferably, when stirring is mechanical or magnetic stirring, the stirring speed is greater than 50 rpm and the stirring time is greater than 1 minute. For example, the stirring speed is between 50 rpm to 1500 rpm, and the stirring time is between 0.5 hours to 5 hours. During the ultrasonic treatment, the ultrasonic power is between 50 W to 500 W, and the time is greater than 0.1 seconds, such as 2 to 200 seconds. During the homogenization treatment, use a high-pressure/ultra-high-pressure homogenizer or a high-shear homogenizer. When using the high-pressure/ultra-high-pressure homogenizer, the pressure is greater than 20 psi, such as 20 psi to 100 psi. When using the high-shear homogenizer, the speed is greater than 1000 rpm, such as 1000 rpm to 5000 rpm. When using microfluidic treatment, the flow rate is greater than 0.01 mL/min, such as 0.1 mL/min to 100 mL/min. Nanoization and/or micronization may be achieved through ultrasonic, or stirring, or homogenization treatment, or microfluidic treatment. Ultrasonic duration, or stirring speed, or homogenization pressure and time can control the size of the prepared nano/micronparticles, it will cause changes in particle size if it is too large or too small.

In the present disclosure, the emulsifier aqueous solution is a polyvinyl alcohol (PVA) aqueous solution, with the third predetermined volume of 5 mL and the third predetermined concentration of 20 mg/mL. The third predetermined volume is adjusted according to its ratio to the second predetermined volume. In the present disclosure, the range between the second and the third predetermined volumes is set from 1:1.1 to 1:1000, preferably may be 2:5. In the procedure of the specific embodiment, to control the size of nanoparticles or micronparticles, the ratio of the second and the third predetermined volumes may be adjusted. Similarly, the values of ultrasonic time or stirring time, the volume of emulsifier aqueous solution, and concentration in this step are all based on the purpose of obtaining nanoparticles or micronparticles with appropriate size.

Step 4, adding the liquid obtained from step 3 to the fourth predetermined volume emulsifier aqueous solution with the fourth predetermined concentration, and stirring until the predetermined stirring conditions are met or directly carrying out subsequent processing without stirring.

In this step, the emulsifier aqueous solution is still PVA.

The fourth predetermined concentration is 5 mg/mL, and the selection of the fourth predetermined concentration is based on obtaining nanoparticles or micronparticles with appropriate size. The selection of the fourth predetermined volume is determined by the ratio of the third predetermined volume to the fourth predetermined volume. In the present disclosure, the ratio of the third predetermined volume to the fourth predetermined volume ranges from 1:1.5 to 1:2000, preferably 1:10. In the procedure of the specific embodiment, to control the size of nanoparticles or micronparticles the ratio of the third and the fourth predetermined volumes may be adjusted.

In the present disclosure, the predetermined stirring condition for this step is until the organic solvent fully volatilizes, that is, the dichloromethane volatilization in step 1 fully volatilizes. Subsequent experiments may also be carried out without stirring.

Step 5, centrifuging the mixture processed in step 4 that meets the predetermined stirring conditions at a speed greater than 100 RPM for more than 1 minute, removing the supernatant, and resuspending the remaining precipitate in the fifth predetermined volume solution with the fifth predetermined concentration and containing water-soluble and/or non-water-soluble components of whole cell components, or resuspending the remaining precipitate in the fifth predetermined volume solution with the fifth predetermined concentration and containing water-soluble and/or non-water-soluble components of whole cell components mixed with adjuvants.

Step 6, centrifuging the mixture processed in step 5 that meets the predetermined stirring conditions at a speed greater than 100 RPM for more than 1 minute, removing the supernatant, and resuspending the remaining precipitate in the sixth predetermined volume of curing treatment reagents or mineralization treatment reagents, centrifuging and washing after a certain period of action, then adding the seventh predetermined volume of positively or negatively charged substances and acting for a certain period.

In some embodiments of the present disclosure, the freeze-drying is not required when resuspending the residue obtained in step 6 in the seventh predetermined volume of charged substances, and subsequent experiments related to the adsorption of cancer cell lysates on the surface of nanoparticles or micronparticles may be directly carried out.

In some embodiments of the present disclosure, room temperature vacuum drying or freeze vacuum drying is performed when resuspending the residue obtained in step 6 in an aqueous solution with freeze-drying protectant, experiments related to the adsorption of the cancer cell lysates on the surfaces of the nanoparticles or the micronparticles are conducted after the freeze-drying.

In the present disclosure, the freeze-drying protectant is trehalose or a mixed solution of mannitol and sucrose. In the present disclosure, the concentration of the drying protectant in this step is 4% by mass. The reason for this setting is not to affect the drying effect in subsequent drying.

Step 7, drying the suspension containing the drying protectant obtained in step 6 and then reserving the dried substance.

Step 8, mixing the eighth predetermined volume of suspension containing nanoparticles resuspended in PBS (or normal saline) obtained in step 6 or resuspending the dried substance containing nanoparticles or micronparticles and drying protectants after drying obtained in step 7 by the eighth predetermined volume of PBS (or normal saline), with the ninth predetermined volume of water-soluble components or original non-water-soluble components dissolved in 8M urea to obtained nanoparticles or micronparticles.

In the present disclosure, the steps of modification and antigen loading in steps 5-8 may be repeated multiple times to increase the antigen loading capacity. Moreover, when adding positively or negatively charged substances, the substances with the same charge may be added multiple times or the substances with different charges may be added alternately.

In some embodiments, when the volume of the resuspended nanoparticle suspension is 10 mL, the volume of the cancer cell lysates or the water-soluble components containing the tumor tissue lysates or the original non-water-soluble components is 0.1-100 mL. In actual use, the volume and ratio of these may be adjusted as needed.

Step 9, mixing the nanoparticles and/or the micronparticles prepared in step 8 with the dendritic cells and incubating for a certain period.

Step 10, collecting the activated dendritic cells from step 9 and reintroducing them into the body for cancer prevention or treatment.

In the present disclosure, the water-soluble components containing the cancer cell lysates or the tumor tissue lysates, or the original non-water-soluble components, as used, containing poly (I:C), manganese adjuvants, BCG vaccines (BCG) or CpG, and the concentration of poly(I:C), calcium adjuvant, BCG or CpG is greater than 0.01 ng/mL.

Furthermore, during the preparation of the vaccine according to the present disclosure, when the activated dendritic cells in vitro, the nanoparticles and/or the micronparticles loaded only with the water-soluble components and the nanoparticles and/or the micronparticles loaded only with the non-water-soluble components, the nanoparticles and/or the micronparticles loaded only with the water-soluble components, the nanoparticles and/or the micronparticles loaded only with the non-water-soluble components, or the nanoparticles and/or the micronparticles loaded simultaneously with both the water-soluble and the non-water-soluble components may be used together.

From the above technical solution, it can be seen that the present disclosure provides a delivery system that utilizes the nanoparticles or the micronparticles to deliver the water-soluble components and/or the non-water-soluble components of the cells, and the micro/nanoparticle system is utilized to activate the dendritic cells in vitro for the prevention and treatment of the cancer. The cell components of related cells or tissues are divided into two parts based on their solubility in pure water, the water-soluble part that is soluble in pure water and the non-water-soluble part that is insoluble in pure water. Both the water-soluble part and the non-water-soluble part are loaded inside micro/nanoparticles, so most of the mutated proteins or peptides produced by cancer in the cell components are loaded inside micro/nanoparticles to active dendritic cells in vitro. The water-soluble and non-water-soluble parts of the cell components encompass all the components of the entire cells. The water-soluble and non-water-soluble parts of the cell components can also be simultaneously dissolved in aqueous solutions containing solubilizers. Wherein proteins, peptides, and genes that are the same as normal cell components and have not mutated will not cause immunoreaction due to the immune tolerance produced during the development of the autoimmune system, while the mutations in genes, proteins, and peptides caused by cancer have immunogenicity and can activate dendritic cells due to the lack of immune tolerance produced during the development of the autoimmune system. The immunogenic substances produced by disease mutations in the whole cell components can be used to activate dendritic cells for cancer prevention, treatment, and recurrence.

Furthermore, at least one type of the cancer cells or the tumor tissues is identical to the target disease type.

Furthermore, when used as the cancer vaccine to prevent and treat cancer, the vaccine described in the present disclosure may be administered multiple times before the onset of cancer, after the onset of cancer, or after surgical removal of the tumor tissues to activate the immune system, thereby delaying cancer progression, treating cancer, or preventing cancer recurrence.

Through the above scheme, the present disclosure has at least the following advantages:

The present disclosure provides a vaccine system for activating dendritic cells in vitro for the prevention and treatment of cancer, based on using nanoscale or micronscale particles to deliver water-soluble and/or non-water-soluble components of cells. The system maximizes the types of antigens that dendritic cells phagocytose and present in vitro and utilizes tumor-specific T cells activated by antigens in whole cell components or the mixtures thereof can prevent or treat cancer.

The above description is only an overview of the technical solution of the present disclosure. To understand the technical means of the present disclosure more clearly and to implement according to the content of the specification, the following are preferred embodiments of the present disclosure in conjunction with detailed descriptions of the drawings.

### BRIEF DESCRIPTIONS OF THE DRAWINGS

In order to facilitate a clearer understanding of the content of the present disclosure, further detailed explanations will be provided below based on specific embodiments of the present disclosure and in conjunction with the drawings.

FIG. 1 is a schematic diagram of a preparation process and an application field of vaccine system of the present disclosure. Among them, a is a schematic diagram of collecting water-soluble components and non-water-soluble components respectively to prepare nanoparticles or micronparticles; b is a schematic diagram of dissolving whole cell components using a solubilized solution containing solubilizers and preparing the nanoparticles or the micronparticles; c is a schematic diagram of using particles prepared in a or b to activate a dendritic cell vaccine and prevent or treat a cancer with the vaccine.

FIG. 2-FIG. 13 respectively show experimental results of tumor growth rates and survival periods in mice using the dendritic cell vaccine for cancer prevention or treatment in Examples 1-12. a. Experimental results of tumor growth rates during using the vaccine to prevent or treat the cancer (n≥8). b. Experimental results of mice survival during the vaccine prevention or treatment of the cancer (n≥8), with mean±standard error (mean±SEM) for each data point. Significant differences in tumor growth inhibition experiments in Figure a are analyzed by ANOVA, while in Figure b, significant differences are analyzed by Kaplan Meier and log-rank tests. *** means there is a significant difference (p<0.005) between the vaccine group and the PBS blank control group: ### means there is a significant difference (p<0.005) when compare with DC vaccine control group stimulated with blank nanoparticle: & means there is a significant difference (p<0.05) between the vaccine group and the control group of dendritic cells directly activated by lysates: && means there is a significant difference (p<0.01) between the vaccine group and the control group of dendritic cells directly activated by lysates: &&& means there is a significant difference in p<0.005 between the vaccine group and the control group of dendritic cells directly activated by lysates; $ means there is a significant difference (p<0.05) between the group of modified nanoparticles activated dendritic cell vaccine and the group of unmodified nanoparticles activated dendritic cell vaccine: $$ means there is a significant difference (p<0.01) between the group of the modified nanoparticle activated dendritic cell vaccine and the group of the unmodified nanoparticle activated dendritic cell vaccine: ★ means there is a significant difference (p<0.05) between the group of the dendritic cell vaccine activated by the nanoparticles loaded with whole cell components and the group of the dendritic cell vaccine activated by the nanoparticles loaded with multiple peptide neoantigens; δ means there is a significant difference (p<0.05) between the group of dendritic cell vaccine activated by the nanoparticles loaded with both cell components and adjuvants and the group of dendritic cell vaccine activated by nanoparticles loaded only with cell components; t means there is a significant difference (p<0.05) between the group of dendritic cell vaccine activated by nanoparticles with target heads and the group of dendritic cell vaccine activated by nanoparticles without the target heads.

### DETAILED DESCRIPTION

The following are further explanations of the present disclosure in conjunction with the drawings and specific embodiments, in order to enable those skilled in the art to better understand and implement the present disclosure. However, the listed embodiments are not intended to limit the present disclosure.

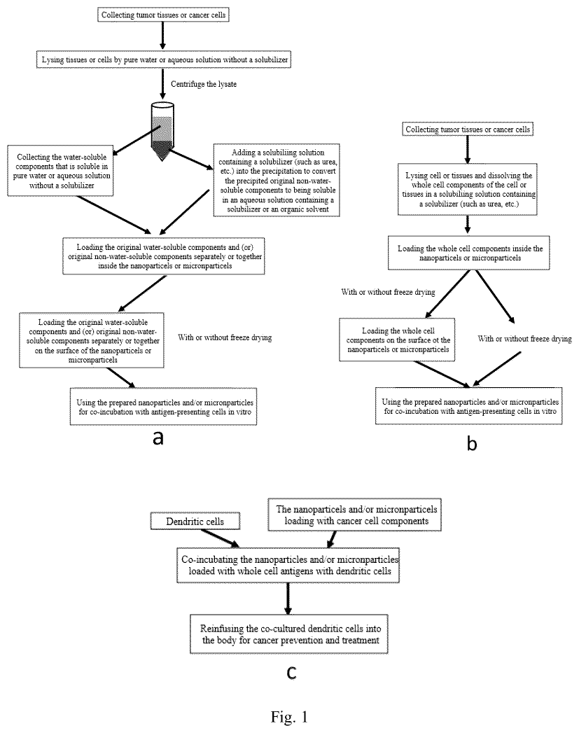

A delivery system of whole cell components or a mixture thereof according to the present disclosure may be used to activate dendritic cells in vitro to prepare a dendritic cell vaccine for preventing and/or treating cancer. A preparation process and an application field are shown in FIG. 1. During the preparation, cells or tissues can be lysed, and water-soluble components and non-water-soluble components may be collected separately to prepare nano/micronparticle systems. Alternatively, solubilizing solution containing a solubilizer may be directly used to lyse the cells or the tissues and dissolve the whole cell components to prepare the nano/micronparticle systems.

The whole cell components according to the present disclosure may be processed before or (and) after lysis, including but not limited to inactivation or (and) denaturation, solidification, biomineralization, ionization, chemical modification, nuclease treatment, etc., and then prepare nanovaccine or micronvaccine. A nanovaccine or a micronvaccine may also be directly prepared without inactivation or (and) denaturation, solidification, biomineralization, ionization, chemical modification, or nuclease treatment before or (and) after cell lysis. In some embodiments of the present disclosure, tumor tissue cells undergo inactivation or (and) denaturation treatment before lysis. In actual use, inactivation or (and) denaturation treatment may also be performed after the cell lysis, alternatively, inactivation or (and) denaturation treatment may also be performed both before and after the cell lysis. The inactivation or (and) denaturation treatment before or (and) after the cell lysis in some embodiments of the present disclosure are ultraviolet radiation and high-temperature heating. In actual use, treatment methods including but not limited to radiation irradiation, high pressure, solidification, biomineralization, ionization, chemical modification, nuclease treatment, collagenase treatment, freeze-drying, etc. may also be used. Those skilled in the art can understand that in practical applications, the technicians may make appropriate adjustments based on specific situations.

### Example 1: Nanoparticles Loaded with Whole Cell Components of Tumor Tissues Activated Dendritic Cells In Vitro and then Reinfused for the Treatment of Melanoma

This example uses mouse melanoma as a cancer model to illustrate how to use nanoparticle system loaded with whole cell components of melanoma tumor tissues to activate dendritic cells in vitro and then reinfuse the dendritic cells to mice for the treatment of melanoma. In this example, B16F10 melanoma tumor tissues are lysed to prepare water-soluble components and non-water-soluble components of the tumor tissues. Then, the organic polymer material PLGA is used as nanoparticle skeleton material, and polyinosinic-polycytidylic acid (poly(I:C)) is used as an immune adjuvant to prepare a nanoparticle system loaded with water-soluble components and non-water-soluble components of tumor tissues by solvent evaporation method. Afterward, the nanoparticle system is co-incubated with dendritic cells (DC) in vitro and the dendritic cells are reinfused into the body to treat melanoma.

### (1) Lysis of the Tumor Tissues and Collection of Various Components

Subcutaneously inoculate 1.5×10⁵ B16-F10 cells on the back of each C57BL/6 mouse, execute the mice and remove the tumor tissues when the tumor grows to a volume of approximately 1000 mm³. Cutting the tumor tissues into pieces and grinding them, adding an appropriate amount of pure water through a cell strainer and repeatedly freezing and—thawing 5 times, accompanied by ultrasound to destroy the lysed cells. After the cells are lysed, centrifuging lysates at a speed of 5000 g for 5 minutes and taking the supernatant to obtain the water-soluble components soluble in pure water. Adding 8M urea to dissolve the precipitate can convert the non-water-soluble components insoluble in pure water into soluble components in 8M urea aqueous solution. The above are the sources of antigen materials for preparing nanoparticle systems.

### (2) Preparation of the Nanoparticle System

In this example, nanovaccines and blank nanoparticles used as controls are prepared by the double emulsion method in solvent evaporation. During the preparation, nanovaccine loaded with water-soluble components of whole cell components and nanoparticles loaded with non-water-soluble components of whole cell components are prepared separately, and then used together. The molecular weight of nanoparticle preparation materials PLGA is 24 KDa-38 KDa, and the immune adjuvant used is poly(I:C), which is only distributed inside nanoparticles. The preparation method is as described above. During the preparation process, antigens are loaded inside nanoparticles by the double emulsion method, after loading the antigens (lysis components) inside, 100 mg of the nanoparticles are centrifuged at 10000 g for 20 minutes, and resuspended in 10 mL of ultrapure water containing 4% trehalose before freeze-drying for 48 hours. Before use, resuspending it with 4 mL of PBS and add 1 mL of tumor tissue lysate components (protein concentration 80 mg/mL), and incubating at room temperature for 10 minutes to obtain the nanoparticle system loaded with lysate both inside and outside. The average particle size of nanoparticles is about 320 nm, and the surface potential of nanoparticles is about-3 mV. Approximately 160 μg of protein or peptide components are loaded onto 1 mg of PLGA nanoparticles, approximately 0.02 mg of poly(I:C) immunoadjuvant is used per 1 mg of PLGA nanoparticles, and the size of the blank nanoparticles is about 300 nm. During the preparation of blank nanoparticles, pure water containing an equal amount of poly(I:C) or 8M urea is used to replace the corresponding the water-soluble components and the non-water-soluble components.

### (3) Preparation of Dendritic Cells

This example takes the preparation of dendritic cells from mouse bone marrow cells as an example to illustrate how to prepare bone marrow-derived dendritic cells (BMDC). Firstly, a 6-8 weeks-old C57 mouse is executed by cervical dislocation, and the tibia and femur of the hind leg are surgically removed and placed in PBS. The muscle tissue around the bone is removed using scissors and forceps. Cutting off both ends of the bone with scissors, then using a syringe to extract the PBS solution. Inserting the needles into the bone marrow cavity from both ends of the bone, and repeatedly rinsing the bone marrow into the culture dishes. Collecting the bone marrow solution, centrifuging at 400 g for 3 minutes, and then adding 1 mL of red blood cell lysis buffer for lysis. Adding 3 mL of RPMI 1640 (10% FBS) medium to terminate the lysis, centrifuging at 400 g for 3 minutes, and discarding the supernatant. Placing the cells in 10 mm culture dishes for culturing, using RPMI 1640 (10% FBS) medium, adding recombinant mouse GM-CSF (20 ng/ml), and culturing at 37° C., 5% CO₂ for 7 days. On the third day, gently shaking the culture flasks and supplementing with the same volume of GM-CSF (20 ng/mL) RPMI 1640 (10% FBS) medium. On the 6th day, changing half of the culture medium. On the 7th day, a small amount of suspended and semi-adherent cells are collected, and flow cytometry analysis showed that when the proportion of CD86⁺CD80⁺ cells in CD11c⁺ cells is between 15-20%, the inducing cultured BMDC could be used for the next experiment.

### (4) Activation of Dendritic Cells

Spreading mouse BMDC onto cell culture plates, adding 5 mL of RPMI 1640 (10% FBS) medium to every 100,000 DC cells, and then adding 30 μg of PLGA nanoparticles loaded with water-soluble components, 30 μg PLGA nanoparticles loaded with non-water-soluble components, and co-incubating with BMDC for 48 hours, then collecting the BMDC, centrifuging at 300 g for 5 minutes and resuspending in PBS after washing twice with phosphate buffer (PBS) for later use.

### (5) Dendritic Cell Cancer Vaccine for Cancer Treatment

Control groups in this study are the PBS group and the blank nanoparticle-stimulated BMDC group. Selected 6-8 weeks-old female C57BL/6 as model mice to prepare melanoma-bearing mice. On day 0, 1.5×10⁵ B16F10 cells are subcutaneously injected into the lower right back of each mouse. The administration plan for dendritic cell vaccine group is as follows: subcutaneous injection of 100 μL vaccine containing 1 million dendritic cells respectively on the 4th, 7th, 10th, 15th, and 20th day after melanoma inoculation. The plan for the PBS control group is as follows: subcutaneous injection of 100 μL PBS respectively on the 4th, 7th, 10th, 15th, and 20th day after melanoma inoculation. Blank nanoparticles control group: subcutaneous injection of 100 μL vaccine containing 500,000 dendritic cells stimulated by blank nanoparticles respectively on the 4th, 7th, 10th, 15th, and 20th day after melanoma inoculation. In the experiment, the volume of mouse tumors is recorded every three days starting from the third day. The volume of tumors is calculated by the formula v=0.52×a×b², wherein v is the volume of the tumor, a is the length of the tumor, and b is the width of the tumor. Due to the ethics of animal experiments, when the volume of tumors exceeds 2000 mm³ in mouse survival test, the mouse is considered dead and euthanized the mouse.

### (6) Experimental Result

As shown in FIG. 2, the tumors of mice in both PBS control group and blank nanoparticle control group grew to be large. Compared with control groups, tumor growth rates of mice in vaccine groups are significantly slower, and tumors of some of the mice disappeared and healed. In summary, dendritic cell vaccine of the present disclosure has good therapeutic effects on melanoma.

### Example 2: Nanoparticles Loaded with Whole Cell Components of Tumor Tissues Activated Dendritic Cell Vaccine In Vitro for the Prevention of Melanoma

This example uses mouse melanoma as a cancer model to illustrate how to use dendritic cell vaccine for cancer prevention. In this example, B16F10 melanoma tumor tissues are lysed to prepare water-soluble components and non-water-soluble components of the tumor tissues. Then, prepare a nanoparticle system loaded with water-soluble components and non-water-soluble components of tumor tissues. In this example, silicification and the addition of charged substances are used to increase the loading of antigens, and only one round of mineralization treatment is performed.

### (1) Lysis of the Tumor Tissues and Collection of Various Components

Subcutaneously inoculating 1.5×10⁵ B16-F10 cells on the back of each C57BL/6 mice, executing mice and removing tumor tissues when the tumor grows to a volume of approximately 1000 mm³. Cutting the tumor tissues into pieces, grinding them, adding collagenase, and incubating them in RPMI 1640 medium for 30 minutes. Then, adding an appropriate amount of pure water through a cell strainer and repeatedly freezing and thawing 5 times, accompanied by ultrasound to destroy the lysed cells. After the cells are lysed, centrifuging the lysates at a speed of 5000 g for 5 minutes and taking the supernatant to obtain the water-soluble components soluble in pure water. Add 8M urea to dissolve the precipitate can convert the non-water-soluble components insoluble in pure water into soluble components in 8M urea aqueous solution. The above are the sources of antigen materials for preparing particles.

### (2) Preparation of the Nanoparticles

In this example, nanoparticles and blank nanoparticles used as controls are prepared by the double emulsion method in solvent evaporation. Appropriate modifications and improvements have been made to the double emulsion method, using two modification methods, low-temperature silicification technology and adding charged substances, to increase the loading of antigens during the preparation of nanoparticles. During the preparation, nanovaccine loaded with water-soluble components of whole cell components and nanoparticles loaded with non-water-soluble components of whole cell components are prepared separately, and then used together. The molecular weight of nanoparticle preparation materials PLGA is 24 KDa-38 KDa, and immune adjuvant used is poly(I:C), which is only distributed inside the nanoparticles. The preparation method is as described above. During the preparation process, antigens are loaded inside the nanoparticles by double emulsion method, after loading antigens (lysis components) inside, 100 mg of nanoparticles are centrifuged at 10000 g for 20 minutes. Then resuspending nanoparticles with 7 mL PBS and mix them with 3 mL PBS solution containing cell lysate (60 mg/mL). Centrifuging at 10000 g for 20 minutes, and resuspend with 10 mL silicate solution (containing 150 mM NaCl, 80 mM tetramethyl orthosilicate, and 1.0 mM HCl, pH 3.0). Fixing at room temperature for 10 minutes, then fixing at −80° C. for 24 hours, washing by centrifugation with ultrapure water, resuspended with 3 mL of PBS containing protamine (5 mg/mL) and polylysine (10 mg/mL) for 10 minutes, then washed by centrifugation at 10000 g for 20 minutes. Resuspending with 10 mL of PBS solution containing cell lysate (50 mg/mL) and incubated for 10 minutes, then centrifuge at 10000 g for 20 minutes and resuspend with 10 mL of ultrapure water containing 4% trehalose. Lyophilized for 48 hours. Before using the particles, resuspending with 7 mL of PBS and adding 3 mL of cancer tissue lysate components containing adjuvant (protein concentration 50 mg/mL) and action at room temperature for 10 minutes to obtain a nanoparticle system modified by frozen silicification and addition of cationic substances and loaded with lysate both inside and outside. The average particle size of the nanoparticles is about 350 nm, and the surface potential of the nanoparticles is about-3 mV. Approximately 300 μg of protein or peptide components are loaded onto 1 mg of PLGA nanoparticles. Approximately 0.02 mg of poly(I:C) immunoadjuvant is used per 1 mg of PLGA nanoparticles, with half inside and half outside.

The preparation process of unmodified nanoparticles is basically the same as that of modified nanoparticles, but does not include low-temperature silicification and the addition of charged substances for treatment. During the preparation process, antigens are loaded inside nanoparticles by the double emulsion method, after loading antigens (lysis components) inside, 100 mg of the nanoparticles are centrifuged at 10000 g for 20 minutes, and resuspended in 10 mL of ultrapure water containing 4% trehalose before lyophilization for 48 hours. Before use, resuspending it with 7 mL of PBS and add 3 mL of tumor tissue lysate components (protein concentration 50 mg/mL), and incubating at room temperature for 10 minutes to obtain nanoparticle system loaded with lysate both inside and outside. The average particle size of nanoparticles is about 320 nm, and the surface potential of nanoparticles is about-5 m V. Approximately 150 μg of protein or peptide components are loaded onto 1 mg of PLGA nanoparticles. Approximately 0.02 mg of poly(I:C) immunoadjuvant is used per 1 mg of PLGA nanoparticles, with half inside and half outside.

The size of the blank nanoparticles is about 300 nm. During the preparation of blank nanoparticles, pure water containing an equal amount of poly(I:C) or 8M urea is used to replace the corresponding water-soluble components and non-water-soluble components.

### (3) Preparation of Dendritic Cells

This example takes the preparation of the dendritic cells from mouse bone marrow cells as an example to illustrate how to prepare bone marrow-derived dendritic cells (BMDC). Firstly, a 6-8 weeks-old C57 mouse is executed by cervical dislocation, and the tibia and femur of the hind leg are surgically removed and placed in PBS. The muscle tissue around the bone is removed using scissors and forceps. Cutting off both ends of the bone with scissors, then using a syringe to extract the PBS solution. Inserting the needles into the bone marrow cavity from both ends of the bone, and repeatedly rinsing the bone marrow into the culture dishes. Collecting the bone marrow solution, centrifuging at 400 g for 3 minutes, and then adding 1 mL of red blood cell lysis buffer for lysis. Adding 3 mL of RPMI 1640 (10% FBS) medium to terminate the lysis, centrifuging at 400 g for 3 minutes, and discarding the supernatant. Placing the cells in 10 mm culture dishes for culturing, using RPMI 1640 (10% FBS) medium, adding recombinant mouse GM-CSF (20 ng/ml), and culturing at 37° C., 5% CO₂ for 7 days. On the third day, gently shaking the culture flasks and supplement with the same volume of GM-CSF (20 ng/mL) RPMI 1640 (10% FBS) medium. On the 6th day, changing half of the culture medium. On the 7th day, a small amount of suspended and semi-adherent cells are collected, and flow cytometry analysis showed that when the proportion of CD86⁺CD80⁺ cells in CD11c⁺ cells is between 15-20%, the inducing cultured BMDC could be used for the next experiment.

### (4) Activation of Dendritic Cells

Spreading mouse BMDC onto cell culture plates, adding 5 mL of RPMI 1640 (10% FBS) medium to every 100,000 DC cells, and then adding 20 μg of PLGA nanoparticles loaded with water-soluble components, 20 μg PLGA nanoparticles loaded with non-water-soluble components, and co-incubating with BMDC for 72 hours, then collecting BMDC, centrifuging at 300 g for 5 minutes and resuspending in PBS after washing twice with PBS for later use.

### (5) Dendritic Cell Cancer Vaccine for Cancer Treatment

Selecting 6-8 weeks-old female C57BL/6 as model mice to prepare melanoma-bearing mice. The administration plan for the dendritic cell vaccine group is as follows: subcutaneous injection of 100 μL dendritic cell vaccine (500,000 dendritic cells) respectively on the 35th, 28th, 21st, 14th, and 7th day before melanoma inoculation. On day 0, 1.5×10⁵ B16F10 cells are subcutaneously injected into the lower right back of each mouse. The plan for PBS control group is as follows: subcutaneous injection of 100 μL PBS respectively on the 35th, 28th, 21st, 14th, and 7th day before melanoma inoculation. On day 0, 1.5×10⁵ B16F10 cells are subcutaneously injected into the lower right back of each mouse. Free lysates control group: subcutaneous injection of 100 μL dendritic cells activated by free lysates respectively on the 35th, 28th, 21st, 14th, and 7th day before melanoma inoculation. On day 0, 1.5×10⁵ B16F10 cells are subcutaneously injected into the lower right back of each mouse. In the experiment, the volume of mouse tumors is recorded every three days starting from the third day. The volume of tumors is calculated by the formula v=0.52×a×b², wherein v is the volume of the tumor, a is the length of the tumor, and b is the width of the tumor. Due to the ethics of animal experiments, when the volume of mouse tumors exceeds 2000 mm³ in mouse survival test, the mouse is considered dead and euthanized the mouse.

### (6) Experimental Result

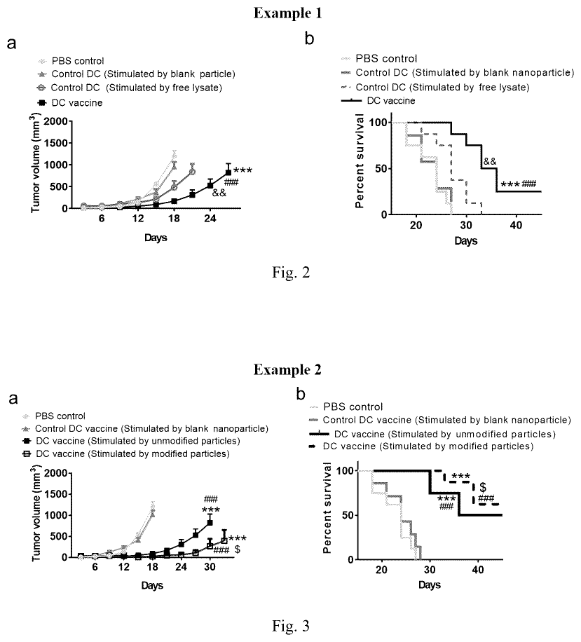

As shown in FIG. 3, the tumors of mice in control group grew faster, while the tumor growth rates of mice immunized with dendritic cell vaccine activated by nanoparticles loaded with antigens are significantly slower. Furthermore, dendritic cell vaccine activated by nanoparticles modified with silicification and charged substances has better preventive effects on melanoma than dendritic cell vaccine group activated by nanoparticles without modification during the preparation process.

### Example 3: Nanoparticles Loaded with Whole Cell Components of Cancer Cells Activated Dendritic Cells for the Prevention of Cancer

In this example, B16F10 melanoma tumor tissues are lysed to prepare water-soluble components and non-water-soluble components of the tumor tissues. Then, the organic polymer material PLGA is used as the nanoparticle skeleton material, and CpG is used as an immune adjuvant to prepare a nanoparticle system loaded with whole cell components of cancer tissues by solvent evaporation method. In this example, silicification, addition of cationic substances and anionic substances are used to increase the loading of antigens, and two rounds of silicification treatment are performed. Nanoparticle system is co-incubated with dendritic cells in vitro and dendritic cells are reinfused to prevent cancer.

### (1) Lysis of Cancer Cells and Collection of Various Components

Collected the cultured B16F10 melanoma cancer cell line and centrifuged at 350 g for 5 minutes. Discarding the supernatant and wash twice with PBS. Then resuspending the cells in ultrapure water and repeatedly freezing and thawing 5 times, accompanied by ultrasound to destroy the lysed cells. After the cells are lysed, centrifuging the lysate at a speed of 3000 g for 6 minutes and taking the supernatant to obtain the water-soluble components soluble in pure water. Adding 8M urea to dissolve the precipitate can convert non-water-soluble components insoluble in pure water into solubilized components in 8M urea aqueous solution. The above are the sources of antigen materials for preparing nanoparticle systems.

### (2) Preparation of Nanoparticle System

In this example, nanoparticles and blank nanoparticles used as controls are prepared by the double emulsion method in solvent evaporation. Appropriate modifications and improvements have been made to the double emulsion method, using two modification methods, low-temperature silicification technology and adding charged substances, to increase the loading of antigens during the preparation of nanoparticles. During preparation, nanovaccine loaded with water-soluble components of whole cell components and nanoparticles loaded with non-water-soluble components of whole cell components are prepared separately, and then used together. The molecular weight of nanoparticle preparation materials PLGA is 7 KDa-17 KDa, and the immune adjuvant used is CpG, which is distributed both inside and on the surface of nanoparticles. The preparation method is as described above. During the preparation process, antigens are loaded inside the nanoparticles by double emulsion method, after loading antigens (lysis components) inside, 100 mg of nanoparticles are centrifuged at 10000 g for 20 minutes. Then resuspending the nanoparticles with 7 mL PBS and mixing them with 3 mL PBS solution containing cell lysate (60 mg/mL). Centrifuging at 10000 g for 20 minutes, and resuspending with 10 mL silicate solution (containing 150 mM NaCl, 80 mM tetramethyl orthosilicate, and 1.0 mM HCl, pH 3.0). Fixing at room temperature for 12 hours, washed by centrifugation with ultrapure water, resuspended with 3 mL of PBS containing polyaspartic acid (10 mg/mL) for 10 minutes, then washed by centrifugation at 12000 g for 18 minutes. Resuspending with 10 mL of PBS solution containing cell lysate (50 mg/mL) for 10 minutes, then centrifuging at 10000 g for 20 minutes. Resuspending with 10 mL silicate solution (containing 150 mM NaCl, 80 mM tetramethyl orthosilicate, and 1.0 mM HCl, pH 3.0). Fixing at room temperature for 12 hours, washed by centrifugation with ultrapure water, resuspending with 3 mL of PBS containing histone (5 mg/mL) and poly arginine (10 mg/mL) for 10 minutes, then washed by centrifugation at 10000 g for 20 minutes. Resuspending with 10 mL of PBS solution containing cell lysate (50 mg/mL) and incubated for 10 minutes, then centrifuge at 10000 g for 20 minutes and resuspending with 10 mL of ultrapure water containing 4% trehalose. Freeze drying for 48 hours. Before using the particles, resuspending with 7 mL of PBS and adding 3 mL of cancer tissue lysate components containing adjuvant (protein concentration 50 mg/mL) and action at room temperature for 10 minutes to obtain nanoparticles loaded with lysates both inside and outside, modified with two rounds of freeze-thaw silicification and the addition of cationic substances and anionic substances. The average particle size of the nanoparticles is about 350 nm, and the surface potential of the nanoparticles is about-3 mV. Approximately 350 μg of protein or peptide components are loaded onto 1 mg of PLGA nanoparticles. Approximately 0.02 mg of CpG immunoadjuvant is used per 1 mg of PLGA nanoparticles, with half inside and half outside.

The preparation process of unmodified nanoparticles is basically the same as that of modified nanoparticles, but does not include low-temperature silicification and the addition of cationic substances and anionic substances for treatment. During the preparation process, antigens are loaded inside the nanoparticles by double emulsion method, after loading antigens (lysis components) inside, 100 mg of the nanoparticles are centrifuged at 10000 g for 20 minutes, and resuspended in 10 mL of ultrapure water containing 4% trehalose before freeze-drying for 48 hours. Before use, resuspending it with 7 mL of PBS and add 3 mL of tumor tissue lysate components (protein concentration 50 mg/mL), and incubating at room temperature for 10 minutes to obtain the nanoparticle system loaded with lysate both inside and outside. The average particle size of the nanoparticles is about 320 nm, and the surface potential of the nanoparticles is about-5 mV. Approximately 160 μg of protein or peptide components are loaded onto 1 mg of PLGA nanoparticles. Approximately 0.02 mg of CpG immunoadjuvant is used per 1 mg of PLGA nanoparticles, with half inside and half outside.

The size of blank nanoparticles is about 300 nm. During the preparation of blank nanoparticles, pure water containing an equal amount of CpG or 8M urea is used to replace the corresponding water-soluble components and non-water-soluble components.

### (3) Preparation of Dendritic Cells

This example takes the preparation of dendritic cells from mouse bone marrow cells as an example to illustrate how to prepare bone marrow-derived dendritic cells (BMDC). Firstly, a 6-8 weeks-old C57 mouse is executed by cervical dislocation, and the tibia and femur of the hind leg are surgically removed and placed in PBS. The muscle tissue around the bone is removed using scissors and forceps. Cutting off both ends of the bone with scissors, then use a syringe to extract the PBS solution. Inserting the needles into the bone marrow cavity from both ends of the bone, and repeatedly rinse the bone marrow into the culture dishes. Collecting the bone marrow solution, centrifuge at 400 g for 3 minutes, and then adding 1 mL of red blood cell lysis buffer for lysis. Adding 3 mL of RPMI 1640 (10% FBS) medium to terminate the lysis, centrifuge at 400 g for 3 minutes, and discarding the supernatant. Placing the cells in 10 mm culture dishes for culturing, using RPMI 1640 (10% FBS) medium, add recombinant mouse GM-CSF (20 ng/mL), and culturing at 37° C., 5% CO₂ for 7 days. On the third day, gently shaking the culture flasks and supplementing with the same volume of GM-CSF (20 ng/mL) RPMI 1640 (10% FBS) medium. On the 6th day, changing half of the culture medium. On the 7 day, a small amount of suspended and semi-adherent cells are collected, and flow cytometry analysis showed that when the proportion of CD86⁺CD80⁺ cells in CD11c⁺ cells is between 15-20%, the inducing cultured BMDC could be used for the next experiment.

### (4) Activation of Dendritic Cells

Spreading mouse BMDC onto cell culture plates, adding 5 mL of RPMI 1640 (10% FBS) medium to every 100,000 DC cells, and then adding 20 μg of PLGA nanoparticles loaded with water-soluble components, 20 μg PLGA nanoparticles loaded with non-water-soluble components, and co-incubating with BMDC for 72 hours, then collecting the BMDC, centrifuge at 300 g for 5 minutes and resuspending in PBS after washing twice with PBS for later use.

### (5) Dendritic Cell Cancer Vaccine for Cancer Treatment

Selecting 6-8 weeks-old female C57BL/6 as model mice to prepare melanoma-bearing mice. The administration plan for the dendritic cell vaccine group is as follows: subcutaneous injection of 100 μL dendritic cell vaccine (1,000,000 dendritic cells) respectively on the 35th, 28th, 21st, 14th, and 7th day before melanoma inoculation. On day 0, 1.5×10⁵ B16F10 cells are subcutaneously injected into the lower right back of each mouse. The plan for PBS control group is as follows: subcutaneous injection of 100 μL PBS respectively on the 35th, 28th, 21st, 14th, and 7th day before melanoma inoculation. On day 0, 1.5×10⁵ B16F10 cells are subcutaneously injected into the lower right back of each mouse. Blank nanoparticles or free lysates control group: subcutaneous injection of 100 μL dendritic cells activated by blank nanoparticles or free lysates respectively on the 35th, 28th, 21st, 14th, and 7th day before melanoma inoculation. On day 0, 1.5×10⁵ B16F10 cells are subcutaneously injected into the lower right back of each mouse. In the experiment, the volume of mouse tumors is recorded every three days starting from the third day. The volume of tumors is calculated by the formula v=0.52×a×b², wherein v is the volume of the tumor, a is the length of the tumor, and b is the width of the tumor. Due to the ethics of animal experiments, when the volume of mouse tumors exceeds 2000 mm³ in mouse survival test, the mouse is considered dead and euthanized the mouse.

### (6) Experimental Result

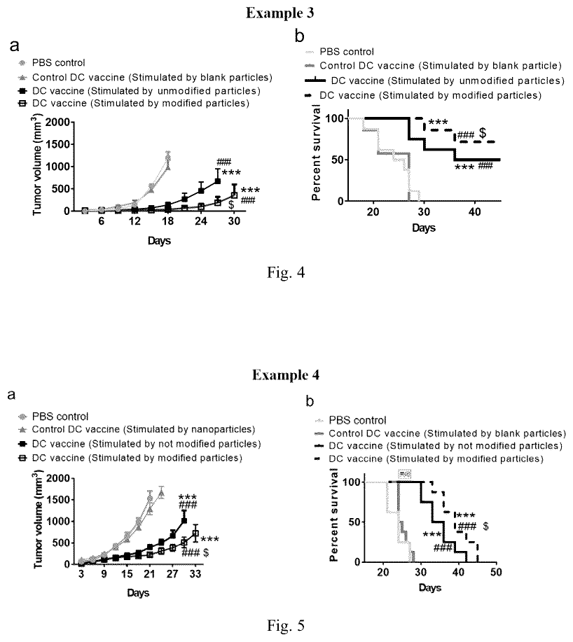

As shown in FIG. 4, the tumors of mice in control group grew faster, while the tumor growth rates of mice immunized with dendritic cell vaccine activated by nanoparticles loaded with antigens are significantly slower. Furthermore, dendritic cell vaccine activated by nanoparticles modified with silicification and charged substances has better preventive effects on melanoma than dendritic cell vaccine group activated by nanoparticles without modification during the preparation process.

### Example 4: Nanoparticles Loaded with Whole Cell Components of Colon Cancer Tumor Tissues and Cancer Cells Activated Dendritic Cells In Vitro for the Treatment of Colon Cancer

This example uses mouse colon cancer as a cancer model to illustrate how to use dendritic cell vaccine for colon cancer treatment. In this example, the MC38 mouse colon cancer cells are used as a cancer model. First, the colon cancer tumor tissues and colon cancer cells are lysed to prepare water-soluble components and non-water-soluble components of the tumor tissues. Then, the organic polymer material PLGA is used as the nanoparticle skeleton material, and the Bacillus Calmette-Guérin vaccine (BCG) is used as an immune adjuvant to prepare nanoparticles. The nanoparticles are used to activate dendritic cells in vitro, and then reinfused dendritic cells to treat colon cancer.

### (1) Lysis of the Tumor Tissues and the Cancer Cells and Collection of Various Components

Subcutaneously inoculating 2×10⁶ MC38 cells on the back of each C57BL/6 mice, executing the mice and removing tumor tissues when the tumor grows to a volume of approximately 1000 mm³. Cutting the tumor tissues into pieces and grinding them, adding an appropriate amount of pure water through a cell strainer and repeatedly freezing-thawing 5 times, accompanied by ultrasound to destroy the lysed cells. After the cells are lysed, centrifuging the lysate at a speed greater than 5000 g for 5 minutes and taking the supernatant to obtain water-soluble components soluble in pure water. Adding 8M urea to dissolve the precipitate can convert non-water-soluble components insoluble in pure water into soluble components in 8M urea aqueous solution.

Collecting cultured MC38 cancer cell line and centrifuging at 350 g for 5 minutes. Discarding the supernatant and wash twice with PBS. Then resuspending the cells in ultrapure water and repeatedly freezing-thawing 5 times, accompanied by ultrasound to destroy the lysed cells. After the cells are lysed, centrifuging lysate at a speed of 3000 g for 6 minutes and taking supernatant to obtain water-soluble components soluble in pure water. Adding 8M urea to dissolve the precipitate can convert non-water-soluble components insoluble in pure water into soluble components in 8M urea aqueous solution.

Mixing water-soluble components from MC38 tumor tissues and MC38 cancer cells with non-water-soluble components dissolved in 8M urea in a ratio of 1:1 as the sources for preparing nanoparticles.

### (2) Lysis of BCG and Collection of Various Components

The lysis method of BCG and collection method of various components are the same as lysis method of cancer cells and collection method of various components.

### (3) Preparation of the Nanoparticles

In this example, nanoparticles and the blank nanoparticles used as controls are prepared by the double emulsion method in solvent evaporation. Appropriate modifications and improvements have been made to double emulsion method. Preparation method is the same as in Example 1, except that water-soluble or non-water-soluble components in Example 1 are replaced with the corresponding mixture in this example.

### (4) Preparation of Dendritic Cells

Same as Example 3.

### (5) Activation of the Dendritic Cells

Same as Example 3.

### (6) Dendritic Cell Cancer Vaccine for Cancer Treatment

Control groups in this study are PBS group and blank nanoparticles or free lysates stimulated BMDC group. Selecting 6-8 weeks-old female C57BL/6 as model mice to prepare melanoma-bearing mice. On day 0, 2×10⁶ MC38 cells are subcutaneously injected into the lower right back of each mouse. The administration plan for dendritic cell vaccine group is as follows: subcutaneous injection of 200 μL vaccine containing 1 million dendritic cells respectively on the 4th, 7th, 10th, 15th, and 20th day after tumor inoculation. The plan for PBS control group is as follows: subcutaneous injection of 200 μL PBS respectively on the 4th, 7th, 10th, 15th, and 20th day after inoculating cancer cells. Blank nanoparticles or free lysates control group: subcutaneous injection of 200 μL vaccine containing 500,000 dendritic cells stimulated by blank nanoparticles respectively on the 4th, 7th, 10th, 15th, and 20th day after tumor inoculation. In the experiment, the volume of mouse tumors is recorded every three days starting from the third day. The volume of tumors is calculated by the formula v=0.52×a×b², wherein v is the volume of the tumor, a is the length of the tumor, and b is the width of the tumor. Due to the ethics of animal experiments, when the volume of mouse tumors exceeds 2000 mm³ in mouse survival test, the mouse is considered dead and euthanized the mouse.

### (7) Experimental Result

As shown in FIG. 5, the tumors of mice in both PBS control group and blank nanoparticle control group grew rapidly. Compared with control groups, tumor growth rates of mice in vaccine groups are significantly slower, and tumors in some mice disappeared and healed. In summary, dendritic cell vaccine of the present disclosure has good therapeutic effects on colon cancer.

### Example 5: Treating Melanoma Using Dendritic Cell Vaccine Activated by Nanoparticles Loaded with Whole Cell Components of Melanoma Tumor Tissues and Lung Cancer Tumor Tissues

This example uses melanoma as a cancer model to illustrate how to use the nanoparticles loaded with whole cell components of melanoma tumor tissues and lung cancer tumor tissues to activate dendritic cells, and then use the cell vaccine for the treatment of melanoma. In this example, the B16F10 melanoma tumor tissues and LLC lung cancer tumor tissues are lysed to prepare water-soluble components and non-water-soluble components of tumor tissues. Then, the organic polymer material PLGA is used as the nanoparticle skeleton material, and the manganese particles and CpG are used as an immune adjuvant to prepare nanoparticles loaded with tumor tissue components by solvent evaporation method. Then, the nanoparticles are used to activate dendritic cells, and the dendritic cell vaccine are used for the treatment of melanoma.

### (1) Lysis of the Tumor Tissues and Collection of Various Components

Subcutaneously inoculating 1.5×10⁵ B16-F10 cells or 2×10⁶ LLC lung cancer cells on the back of each C57BL/6 mouse, executing the mice and remove tumor tissues when the tumor grows to a volume of approximately 1000 mm³. The methods of tumor lysis and collection of various components are the same as in Example 1. Mixing water-soluble components from B16-F10 tumor tissues and LLC lung cancer tumor tissues with non-water-soluble components dissolved in 8M urea in a ratio of 1:1 as the antigen sources for preparing nanoparticles.

(2) Preparation of the Nanoparticles Loaded with Antigens

In this example, nanoparticles and blank nanoparticles used as controls are prepared by the double emulsion method in solvent evaporation. Appropriate modifications and improvements have been made to double emulsion method, using two modification methods, low-temperature silicification technology and adding charged substances, to increase the loading of antigens during the preparation of nanoparticles. During the preparation, nanovaccines loaded with water-soluble components of whole cell components and nanoparticles loaded with non-water-soluble components of whole cell components are prepared separately, and then used together. The molecular weight of nanoparticle preparation materials PLGA is 24 KDa-38 KDa, and the immune adjuvant used are manganese colloidal particles and CpG. The manganese particles are distributed inside the nanoparticles, while the CpG is distributed on the surface of the nanoparticles. Firstly, preparing the manganese adjuvant, and then mixing it with water-soluble components or non-water-soluble components of whole cell components as the first aqueous phase to prepare nanoparticles internally loaded with antigens and adjuvants using double emulsion method. To prepare the manganese adjuvant, firstly adding 1 mL of 0.3 M Na₃PO₄ solution to 9 mL of normal saline, then adding 2 mL of 0.3 M MnCl₂ solution, and leaving it overnight to obtain Mn₂OHPO₄ colloidal manganese adjuvant, with a particle size of approximately 13 nM. Then, the manganese adjuvant is mixed with water-soluble components or non-water-soluble components (60 mg/mL) of whole cell components in a volume ratio of 1:3, and then antigen and manganese adjuvant are loaded into the nanoparticles by the double emulsion method. After loading antigens (lysis components) inside, 100 mg of nanoparticles are centrifuged at 10000 g for 20 minutes. Then resuspending the nanoparticles with 7 mL PBS and mix them with 3 mL PBS solution containing cell lysate (50 mg/mL). Centrifuging at 10000 g for 20 minutes, and resuspending with 10 mL silicate solution (containing 150 mM NaCl, 80 mM tetramethyl orthosilicate, and 1.0 mM HCl, pH 3.0). Fixing at room temperature for 10 minutes, then fixing at −80° C. for 24 hours, washed by centrifugation with ultrapure water, resuspending with 3 mL of PBS containing histone (5 mg/mL) and polyarginine (10 mg/mL) for 10 minutes, then washed by centrifugation at 10000 g for 20 minutes. Resuspending with 10 mL of PBS solution containing cell lysate (50 mg/mL) and incubating for 10 minutes, then centrifuging at 10000 g for 20 minutes and resuspending with 10 mL of ultrapure water containing 4% trehalose. Freeze drying for 48 hours. Before incubating nanoparticles and dendritic cells, resuspending with 7 mL of PBS and add 3 mL of cancer tissue lysate components containing CpG adjuvant (protein concentration 50 mg/mL) and react at room temperature for 10 minutes to obtain a nanoparticle system modified by frozen silicification and addition of cationic substances and loaded with lysate both inside and outside. The average particle size of the nanoparticles is about 360 nm, and the surface potential of the nanoparticle is about-3 mV. Approximately 320 μg of protein or peptide components are loaded onto 1 mg of PLGA nanoparticles. Approximately 0.01 mg of CpG adjuvant is used inside and outside per 1 mg of PLGA nanoparticles.

The size of blank nanoparticles is about 300 nm. During the preparation of blank nanoparticles, pure water containing an equal amount of manganese adjuvant and CpG adjuvant, or 8M urea is used to replace the corresponding water-soluble components and non-water-soluble components.

### (4) Preparation of the Dendritic Cells

Same as Example 1.

### (5) Activation of the Dendritic Cells

Same as Example 1.

### (6) The Dendritic Cell Cancer Vaccine for Cancer Treatment

The control groups in this study are PBS group and blank nanoparticles or free lysates stimulated BMDC group. Selecting 6-8 weeks-old female C57BL/6 as model mice to prepare melanoma-bearing mice. On day 0, 1.5×10⁵ B16F10 cells are subcutaneously injected into the lower right back of each mouse. The administration plan for dendritic cell vaccine group is as follows: subcutaneous injection of 50 μL vaccine containing 500,000 dendritic cells respectively on the 4th, 7th, 10th, 15th, and 20th day after melanoma inoculation. The plan for PBS control group is as follows: subcutaneous injection of 50 μL PBS respectively on the 4th, 7th, 10th, 15th, and 20th day after melanoma inoculation. Blank nanoparticles or free lysates control group: subcutaneous injection of 50 μL vaccine containing 500,000 dendritic cells stimulated by blank nanoparticles or free lysates respectively on the 4th, 7th, 10th, 15th, and 20th day after melanoma inoculation. In the experiment, the volume of mouse tumors is recorded every three days starting from the third day. The volume of tumors is calculated by the formula v=0.52×a×b², wherein v is the volume of tumor, a is the length of tumor, and b is the width of tumor. Due to the ethics of animal experiments, when the volume of mouse tumors exceeds 2000 mm³ in mouse survival test, the mouse is considered dead and euthanized the mouse.

### (7) Experimental Result

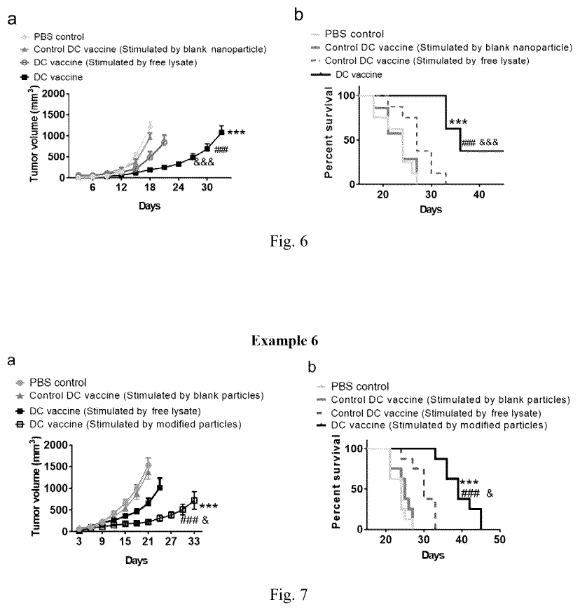

As shown in FIG. 6, tumors of mice in both PBS control group and blank nanoparticle control group grew rapidly. Compared with control groups, tumor growth rates of mice in vaccine groups are significantly slower, and tumors in some mice disappeared and healed. In summary, dendritic cell vaccine of the present disclosure has good therapeutic effects on melanoma.

### Example 6: Treating Colon Cancer Using Dendritic Cell Vaccine Activated by Micronparticles Loaded with Water-Soluble Components of Colon Cancer and Lung Cancer Cells Both Inside and on the Surface of Micronparticles

This example illustrates how to prepare a micronparticle system that is only loaded with water-soluble components of colon cancer and lung cancer cell components. In this example, the MC38 colon cancer tumor tissues and LLC lung cancer tumor tissues are lysed to prepare water-soluble components and non-water-soluble components of the tumor tissues. Then, the organic polymer material PLGA is used as the nanoparticle skeleton material, and the manganese particles and poly(I:C) are used as an immune adjuvant to prepare micronparticles loaded with water-soluble components of whole cells. Then, the particle system is used to activate dendritic cells in vitro and then reinfused for the treatment of colon cancer.

### (1) Lysis of MC38 Colon Cancer Tumor Tissues and LLC Lung Cancer Cells and Collection of Various Components

Collection and lysis methods for tumor tissues and cancer cells, as well as collection methods for water-soluble components and non-water-soluble components, are the same as above. The water-soluble components of the two lysates derived from colon cancer tumor tissues and lung cancer cells are mixed in a ratio of 3:1 as antigen source for preparing the micronparticle system.

### (2) Preparation of Micronparticle System

In this example, the micronparticle and blank micronparticles used as controls are prepared by double emulsion method in solvent evaporation. The molecular weight of micronparticle preparation materials PLGA is 38 KDa-54 KDa, and the immune adjuvants used are manganese particles and poly(I:C). The manganese particles are distributed inside the micronparticles, while the poly(I:C) is distributed on the surface of the micronparticles. During the preparation, firstly preparing the manganese adjuvant, and then mixing with water-soluble components or non-water-soluble components of whole cell components as the first aqueous phase to prepare micronparticles internally loaded with antigens and adjuvants using double emulsion method. To prepare the manganese adjuvant, firstly adding 0.05 mL of 0.6 M Na₃PO₄ solution to 95 mL of normal saline, then adding 0.1 mL of 0.3 M MnCl₂ solution to obtain Mn₂OHPO₄ colloidal manganese adjuvant, with a particle size of approximately 20 nM. Then, the manganese adjuvant is mixed with water-soluble components (60 mg/mL) of whole cell components in a volume ratio of 1:4, and antigens and manganese adjuvant are loaded into the micronparticles by double emulsion method. After loading the antigens (lysis components) inside, 100 mg of the nanoparticles are centrifuged at 10000 g for 20 minutes. Then resuspending the micronparticles with 8 mL PBS and adding 2 mL of lysate components containing poly(I:C) adjuvant (protein concentration 80 mg/mL) and react at room temperature for 10 minutes to obtain a micronparticle system. The average particle size of the micronparticle is about 1.5 μm, and the surface potential of the micronparticle is about-4 mV. Approximately 200 μg of protein or peptide components are loaded onto 1 mg of PLGA micronparticles. Approximately 0.01 mg of poly(I:C) adjuvant is used inside and outside per 1 mg of PLGA micronparticles.

The size of blank micronparticle is about 1.4 μm. During the preparation of blank micronparticles, pure water containing an equal amount of manganese adjuvant and poly(I:C) adjuvant is used to replace the corresponding water-soluble components.

### (4) Preparation of the Dendritic Cells

Same as Example 1.

### (5) Activation of Dendritic Cells

Same as Example 1. During co-incubation, 20 ng/ml GM-CSF and 20 ng/ml IL-2 are added to the cell culture medium.

### (6) Dendritic Cell Cancer Vaccine for Cancer Treatment

Control groups in this study are PBS group and blank micronparticles or free lysates group. Selecting 6-8 weeks-old female C57BL/6 as model mice to prepare mice with colon cancer. On day 0, 2×10⁶ MC38 cells are subcutaneously injected into the lower right back of each mouse. The administration plan for dendritic cell vaccine group is as follows: subcutaneous injection of 100 μL vaccine containing 1,000,000 dendritic cells respectively on the 3rd, 6th, 9th, 12th, and 18th day. The plan for the PBS control group is as follows: subcutaneous injection of 100 μL PBS respectively on the 3rd, 6th, 9th, 12th, and 18th day. Blank micronparticles or free lysates control group: subcutaneous injection of 100 μL dendritic cells (1,000,000 dendritic cells) stimulated by blank micronparticles or free lysates respectively on the 3rd, 6th, 9th, 12th, and 18th day. In the experiment, the volume of mouse tumors is recorded every three days starting from the third day. The volume of tumors is calculated by the formula v=0.52×a×b², wherein v is the volume of the tumor, a is the length of the tumor, and b is the width of the tumor. Due to the ethics of animal experiments, when the volume of mouse tumors exceeds 2000 mm³ in mouse survival test, the mouse is considered dead and euthanized the mouse.

### (4) Experimental Result

As shown in FIG. 7, compared with control groups, tumor growth rates of mice in the vaccine groups are significantly slower. Dendritic cell vaccine activated in vitro by micronparticles loaded with water-soluble components have therapeutic effects on colon cancer.

### Example 7: Prevention of Breast Cancer Using Dendritic Cells Activated In Vitro by Micronparticles Loaded with Breast Cancer Cells Dissolved by 6M Guanidine Hydrochloride

This example uses 4T1 mouse triple-negative breast cancer as the cancer model to illustrate how to use 6M guanidine hydrochloride to solubilize whole cell component and prepare the micronparticle system loaded with whole cell components, and use this micronparticle system to activate dendritic cells in vitro for the prevention of breast cancer. In this example, breast cancer cells are first inactivated and denatured, and whole cell components are solubilized after the cancer cells are lysed by 6M guanidine hydrochloride. Then PLGA is used as the micronparticle skeleton material, CpG and poly(I:C) are used as an immune adjuvant to prepare the micronparticle system loaded with whole cell components by solvent evaporation method. The micronparticle system is used to activate dendritic cells in vitro to prevent breast cancer.

### (1) Lysis of Cancer Cells and Collection of Various Components

Centrifuging the cultured 4T1 cells at 400 g for 5 minutes, then washing twice with PBS and resuspending in ultrapure water. Obtained cancer cells are inactivated and denatured by ultraviolet and high-temperature heating respectively, and then breast cancer cells are lysed with an appropriate amount of 6M guanidine hydrochloride, and the lysates are solubilized as the source of materials for preparing the particle system.

### (2) Preparation of the Micronparticle System

In this example, the micronparticle and blank micronparticle used as controls are prepared by double emulsion method in solvent evaporation. The molecular weight of micronparticle preparation materials PLGA is 38 KDa-54 KDa, and the immune adjuvants used are CpG and poly(I:C). During the preparation, appropriate modifications and improvements are made to double emulsion method. In the process of preparing micronparticles, double emulsion method is first used to prepare micronparticles loaded with antigens and adjuvants inside. After loading the antigens and adjuvants inside, 100 mg of the micronparticles are centrifuged at 10000 g for 20 minutes and resuspended in 10 mL of ultrapure water containing 4% trehalose before freeze-drying for 48 hours. Before injecting the micronparticle system, resuspending it with 8 mL of PBS and adding 2 mL of cancer cell lysate components (protein concentration 80 mg/mL), and incubating at room temperature for 10 minutes to obtain the required micronparticle system. The average particle size of the micronparticle is about 1.5 μm, and the surface potential of the micronparticle is about-4 m V. Approximately 140 μg of protein or peptide components are loaded onto 1 mg of PLGA micronparticles. The size of blank micronparticle is about 1.4 μm. During the preparation of blank micronparticles, 6M guanidine hydrochloride containing an equal amount of CpG and poly(I:C) adjuvant is used to replace the corresponding cell components.

### (3) Preparation of Dendritic Cells

Same as Example 1.

### (4) Activation of Dendritic Cells

Same as Example 1.

### (5) Micronparticle System for Cancer Prevention

Selecting 6-8 weeks-old female BALB/c to prepare mice with 4T1. The vaccine prevention group subcutaneously injected 100 μL dendritic cell vaccine (1,000,000 dendritic cells) respectively on the 35th, 28th, 21st, 14th, and 7th day before tumor inoculation. On day 0, 4×10⁵ 4T1 cells are subcutaneously injected into the lower right back of each mouse. PBS control group subcutaneously injected 100 μL PBS respectively on the 35th, 28th, 21st, 14th, and 7th day before tumor inoculation. On day 0, 4×10⁵ 4T1 cells are subcutaneously injected into the lower right back of each mouse. Blank micronparticles or free lysates control group subcutaneously injected dendritic cells (1,000,000) stimulated by blank lysates or blank micronparticles respectively on the 35th, 28th, 21st, 14th, and 7th day before tumor inoculation. On day 0, 4×10⁵ 4T1 cells are subcutaneously injected into the lower right back of each mouse. In the experiment, the volume of mouse tumors is recorded every three days starting from the third day. The volume of tumors is calculated by the formula v=0.52×a×b², wherein v is the volume of the tumor, a is the length of the tumor, and b is the width of the tumor. The mouse is considered dead and euthanized the mouse when the volume of mouse tumors exceeded 2000 mm³ in mouse survival test.

### (6) Experimental Result

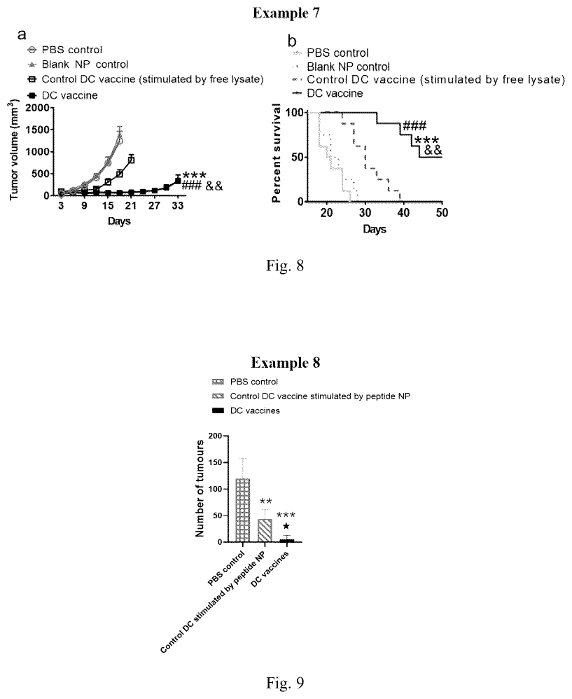

As shown in FIG. 8, compared with control groups, the tumor growth rates of mice in the vaccine groups are significantly slower and the survival periods of mice are significantly prolonged. It can be seen that the vaccine of the present disclosure has preventive effect on breast cancer.

### Example 8: Prevention of Cancer Metastasis Using Dendritic Cells Activated by Nanoparticle Systems Loaded with Whole Cell Components of Tumor Tissues and Cancer Cells

This example uses mouse melanoma as a mouse lung cancer metastasis model to illustrate the use of dendritic cell vaccine to prevent cancer metastasis. In practical application, the specific dosage form, adjuvant, administration time, administration frequency, and administration plan may be adjusted according to situations. In this example, mouse melanoma tumor tissues and cancer cells are lysed with 8M urea and solubilized. Then, the lysis components of tumor tissues and cancer cells are loaded into a nanoparticle system in a mass ratio of 1:4, and dendritic cells are activated by the particle system to prevent cancer metastasis. In this example, nanoparticles loaded with four types of peptide neoantigens, B16-M20 (Tubb3, FRRKAFLHWYTGEAMDEMEFTEAESNM), B16-M24 (Dag1, TAVITPPTTTTKKARVSTPKPATPSTD), B16-M46 (Actn4, NHSGLVTFQAFIDVMSRETTDTDTADQ) and TRP2: 180-188 (SVYDFFVWL), are used as control nanoparticles to analyze the effects of nanoparticles loaded with whole cell antigens and nanoparticles loaded with multiple peptide neoantigens in preparation of dendritic cell vaccine.

### (1) Lysis of Tumor Tissues and Cancer Cells

Collected mouse B16F10 melanoma tumor tissues and cultured cancer cells, then used 8M urea to lyse and dissolve whole cell components of tumor tissues and cancer cells and then mixed tumor tissue components and cancer cell components in a mass ratio of 1:4.

### (2) Preparation of the Nanoparticle System

In this example, the nanoparticle system and blank nanoparticles used as controls are prepared by double emulsion method in solvent evaporation. The molecular weight of nanoparticle preparation materials PLGA is 24 KDa-38 KDa, and the immune adjuvants used are CpG and CaCl₂) and the adjuvants are distributed inside the nanoparticles. The preparation method is as described above. During the preparation process, antigens are loaded inside the nanoparticles by double emulsion method, after loading antigens (lysis components) inside, 100 mg of the nanoparticles are centrifuged at 10000 g for 20 minutes, and resuspended in 10 mL of ultrapure water containing 4% trehalose before freeze-drying for 48 hours. The average particle size of the nanoparticles is about 320 nm. Approximately 160 μg of protein or peptide components are loaded onto 1 mg of PLGA nanoparticles. The preparation method of control nanoparticles loaded with multiple antigen peptides is the same as above. The size of control nanoparticles is about 310 nm, and about 150 μg of antigen peptides are loaded onto 1 mg of PLGA nanoparticles.

### (3) Preparation of Dendritic Cells

Same as Example 1.

### (4) Activation of Dendritic Cells

Same as Example 1.

### (5) Dendritic Cell Cancer Vaccine for Preventing Cancer Metastasis

Selecting 6-8 weeks-old female C57BL/6 as model mice to prepare tumor bearing mice. The vaccine group subcutaneously injected 100 μL dendritic cells (1,000,000) respectively on the 35th, 28th, 21st, 14th, and 7th day before tumor inoculation. PBS control group subcutaneously injected 100 μL PBS respectively on the 35th, 28th, 21st, 14th, and 7th day before tumor inoculation. Control vaccine group subcutaneously injected 100 μL dendritic cells (1,000,000) stimulated by control nanoparticles loaded with peptide antigens respectively on the 35th, 28th, 21st, 14th, and 7th day before tumor inoculation. On day 0, 3×10⁵ B16F10 melanoma cells are subcutaneously injected into tail vein of each mouse. On the 15th day, the mice are euthanized and their lungs are removed. The number of cancer blocks formed by melanoma metastasis in the mice's lungs is observed.

### (6) Experimental Result

As shown in FIG. 9, compared with control groups, the number of cancer metastases in vaccine group mice is significantly reduced. Moreover, dendritic cell vaccine activated by nanoparticles loaded with whole cell components are more effective than dendritic cell vaccine activated by nanoparticles loaded with several antigen peptides. This indicates that dendritic cell vaccine activated in vitro by nanoparticles loaded with whole cell components as described in the present disclosure can effectively prevent cancer metastasis.

### Example 9: Treating Pancreatic Cancer Using Nanoparticles Loaded with Lysates of Pancreatic Cancer Tumor Tissues and Colon Cancer Tumor Tissues Both Inside and Outside

This example uses mouse pancreatic cancer as a cancer model to illustrate the use of dendritic cell vaccine to treat cancer. In this example, the tumor tissues of mouse Pan02 pancreatic cancer and the tumor tissues of MC38 colon cancer are loaded to nanoparticles in a ratio of 2:1.

First, the tumor tissues of mouse pancreatic cancer and colon cancer are obtained and lysed to prepare water-soluble components and original non-water-soluble components dissolved in 6M guanidine hydrochloride. When preparing the particles, the water-soluble component is a mixture of water-soluble components of pancreatic cancer tumor tissues and water-soluble components of colon cancer tumor tissues in a ratio of 2:1. The water-insoluble component is a mixture of the non-water-soluble components of pancreatic cancer tumor tissues and the non-water-soluble components of colon cancer tumor tissues in a ratio of 2:1. PLGA is used as the framework material of nanoparticles, and nanoparticles are prepared without adding any adjuvant. The nanoparticles are used to activate dendritic cells and reinfused into the body to treat Pan02 pancreatic cancer tumor ibearing mice.

### (1) Lysis of the Tumor Tissues and Collection of Various Components

Subcutaneously inoculate 2×10⁶ MC38 colon cancer cells or 1×10⁶ LLC Pan02 pancreatic cancer cells under the armpit of each C57BL/6 mouse, execute the mice and remove tumor tissues when the tumor grows to a volume of approximately 1000 mm³. The lysis method and collection method of various components are the same as in Example 1, except that 6M guanidine hydrochloride is used instead of 8M urea to dissolve the non-water-soluble components.

### (2) Preparation of the Nanoparticles

In this example, the preparation method of nanoparticles is the same as in Example 1, except that adjuvants are not used.

### (3) Preparation of Dendritic Cells

Same as Example 1.

### (4) Activation of Dendritic Cells

Same as Example 1, but 20 ng/ml of GM-CSF is added to the cell culture medium during the incubation process.

### (5) The Vaccine Used for Cancer Treatment

Selecting 6-8 weeks-old female C57BL/6 to prepare mice with pancreatic cancer. On day 0, 1×10⁶ Pan02 cells are subcutaneously injected into the lower right back of each mouse. The administration plan for dendritic cell vaccine group is as follows: subcutaneous injection of 100 μL vaccine containing 1,000,000 dendritic cells respectively on the 3rd, 6th, 9th, 12th, and 18th day. The plan for PBS control group is as follows: subcutaneous injection of 100 μL PBS respectively on the 3rd, 6th, 9th, 12th, and 18th day. Blank nanoparticles or free lysates control group: subcutaneous injection of 100 μL dendritic cells (1,000,000 dendritic cells) stimulated by blank nanoparticles or free lysates respectively on the 3rd, 6th, 9th, 12th, and 18th day. In the experiment, the volume of mouse tumors is recorded every three days starting from the third day. The volume of tumors is calculated by formula v=0.52×a×b², wherein v is the volume of tumor, a is the length of tumor, and b is width of the tumor. Due to the ethics of animal experiments, when the volume of mouse tumors exceeds 2000 mm³ in mouse survival test, the mouse is considered dead and euthanized the mouse.

### (6) Experimental Result

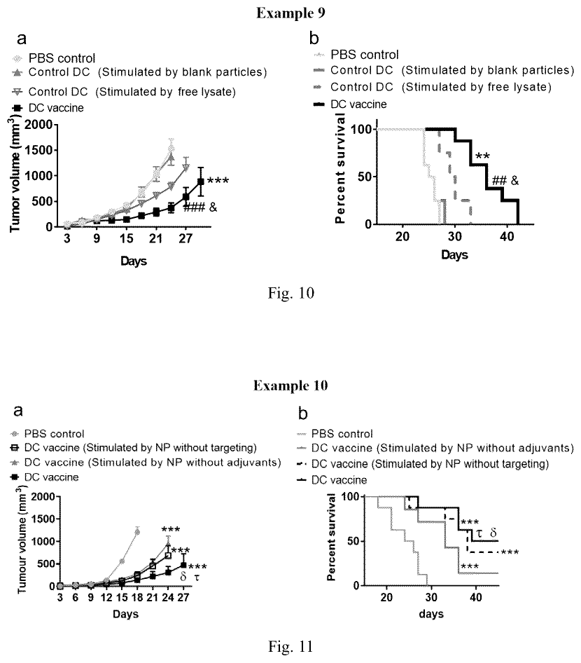

As shown in FIG. 10, compared with control groups, the tumor growth rates in vaccine groups are significantly slower and the survival periods of mice are significantly prolonged. It can be seen that the nanoparticles loaded with cell components of cancer tissues but without adjuvants can activate dendritic cells in vitro and use such dendritic cells as vaccine to treat pancreatic cancer.

### Example 10: Prevention of Cancer Metastasis Using Dendritic Cells Activated by Nanoparticles Loaded with Whole Cell Components of Cancer Cells Target Modified with Mannose

This example uses the mouse melanoma model to illustrate how to use dendritic cell vaccine to prevent cancer. In practical application, the specific dosage form, adjuvant, administration time, administration frequency, and administration plan may be adjusted according to situations. In this example, mouse melanoma cancer cells are lysed with 8M urea and dissolved. Then, the lysates of cancer cells are loaded into a nanoparticle system. The nanoparticle system may enter dendritic cells through the uptake of mannose receptors on the surface of dendritic cells.

### (1) Lysis of the Cancer Cells

Collected the cultured cancer cells, then used 8M urea to lyse and dissolve the whole cell components of cancer cells.

### (2) Preparation of the Nanoparticle System

In this example, the nanoparticle system and the nanoparticles loaded with only cell components but not adjuvants as controls are prepared by double emulsion method in solvent evaporation. The nanoparticle preparation materials used are PLGA and mannose-modified PLGA, with a ratio of 4:1 and molecular weights of 7 KDa-17 KDa for both. The immune adjuvant is CpG and is loaded inside the nanoparticles. The preparation method is as described above. During the preparation process, the cell components are loaded inside the nanoparticles by double emulsion method. After loading the cell components inside, 100 mg of the nanoparticles are centrifuged at 10000 g for 20 minutes and resuspended in 10 mL of ultrapure water containing 4% trehalose, then freeze-dried for 48 hours for later use. The average particle size of nanoparticles with target heads (loaded adjuvants) and without target heads (loaded adjuvants) is about 320 nm. Approximately 60 μg of protein or peptide components are loaded onto 1 mg of PLGA nanoparticles. The particle size of control nanoparticles without adjuvants but with target heads is also about 320 nm. When preparing, equal amounts of cell components are used but did not contain any immune adjuvants. Approximately 60 μg of protein or peptide components are loaded onto 1 mg of PLGA nanoparticles.

### (3) Preparation of Dendritic Cells

Same as Example 1.

### (4) Activation of Dendritic Cells

Same as Example 1.

### (5) Dendritic Cell Cancer Vaccine for Preventing Cancer

Selecting 6-8 weeks-old female C57BL/6 as model mice to prepare tumor-bearing mice. The vaccine group subcutaneously injected 100 μL dendritic cells (1,000,000) respectively on the 35th, 28th, 21st, 14th, and 7th day before tumor inoculation. PBS control group subcutaneously injected 100 μL PBS respectively on the 35th, 28th, 21st, 14th, and 7th day before tumor inoculation. On day 0, 1.5×10⁵ B16F10 melanoma cells are subcutaneously injected into each mouse. In the experiment, the volume of mouse tumors is recorded every three days starting from the third day. The volume of tumors is calculated by tformula v=0.52×a×b², wherein v is the volume of tumor, a is the length of tumor, and b is the width of tumor. Due to the ethics of animal experiments, when the volume of mouse tumors exceeds 2000 mm³ in mouse survival test, the mouse is considered dead and euthanized the mouse.

### (6) Experimental Result

As shown in FIG. 11, compared with control groups, tumor growth rates of mice in vaccine groups are significantly slower. Moreover, dendritic cell vaccine activated by nanoparticles containing target heads are more effective than dendritic cell vaccine activated by nanoparticles without target heads, and dendritic cell vaccine activated by nanoparticles containing immune adjuvants are better than dendritic cell vaccine activated by nanoparticles without immune adjuvants. This indicates that vaccines described in the present disclosure can prevent cancer, and the addition of target heads and adjuvants helps activate dendritic cell vaccine in vitro with nanoparticles.

### Example 11: Prevention of Liver Cancer Using Dendritic Cells Activated In Vitro by Nanoparticles Loaded with Whole Cell Components of Cancer Cells and Adjuvante BCG

This example uses BCG as an immune adjuvant to illustrate how to prepare nanoparticles loaded with whole cell components of liver cancer cells and use the particles to activate dendritic cells in vitro to prevent liver cancer. In this example, PLGA is used as nanoparticle skeleton material, and BCG is used as an immune adjuvant to prepare the nanoparticle system by a solvent evaporation method. Then the particle system is used to activate dendritic cells and prevent liver cancer.

### (1) Lysis of Cancer Cells and Collection of Various Components

In this example, the lysis of cancer cells and collection of lysate are the same as above.

### (1) Lysis of Cancer Cells and Collection of Various Components

In this example, methods of lysis of cancer cells, and collection and solubilization methods of lysates are the same as in Example 1, except that tumor tissue is replaced by cancer cells.

### (2) Preparation of the Nanoparticle System

In this example, the preparation method of nanoparticles and the materials used are the same as in Example 1. However, in this example, the immune adjuvant poly(I:C) encapsulated in the nanoparticles is replaced by BCG.

### (3) Preparation of Dendritic Cells

Same as Example 1.

### (4) Activation of Dendritic Cells

Same as Example 1.

### (5) Dendritic Cell Cancer Vaccine for Preventing Cancer

Selecting 6-8 weeks-old female C57BL/6 as model mice to prepare tumor-bearing mice. The vaccine group subcutaneously injected 100 μL dendritic cells (1,000,000) respectively on the 35th, 28th, 21st, 14th, and 7th day before tumor inoculation. PBS control group subcutaneously injected 100 μL PBS respectively on the 35th, 28th, 21st, 14th, and 7th day before tumor inoculation. The lysate group subcutaneously injected 100 μL dendritic cells (1,000,000) stimulated by free lysates respectively on the 35th, 28th, 21st, 14th, and 7th day before tumor inoculation. On day 0, 2×10⁶ Hepa 1-6 liver cancer cells are subcutaneously injected into each mouse. In the experiment, the volume of mouse tumors is recorded every three days starting from the third day. The volume of tumors is calculated by formula v=0.52×a×b², wherein v is the volume of tumor, a is the length of tumor, and b is the width of tumor. Due to the ethics of animal experiments, when the volume of mouse tumors exceeds 2000 mm³ in mouse survival test, the mouse is considered dead and euthanized the mouse.

### (6) Experimental Result

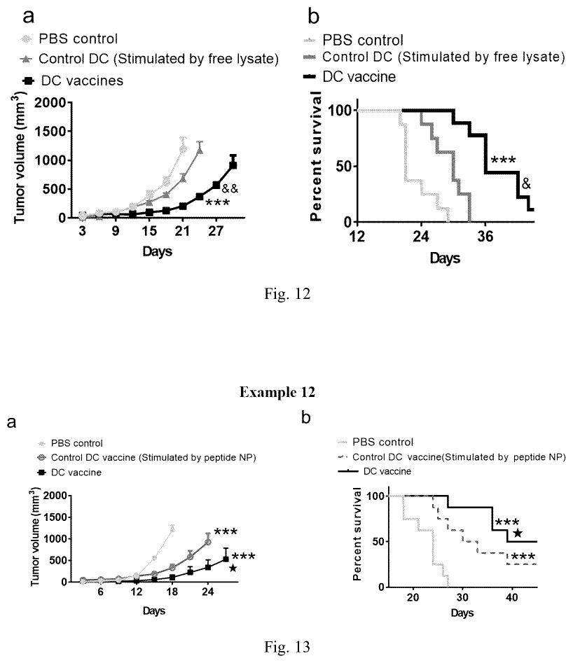

As shown in FIG. 12, compared with control groups, tumor growth rates of mice in vaccine administration groups are significantly slower and the survival periods of mice are significantly prolonged. It can be seen that the vaccine of the present disclosure can prevent liver cancer.

### Example 12: Prevention of Cancer Using Dendritic Cells Activated by Nanoparticle Systems Loaded with Whole Cell Components of Tumor Tissues and Cancer Cells

This example uses melanoma mouse model to illustrate the use of dendritic cell vaccine to prevent cancer metastasis. In practical application, the specific dosage form, adjuvant, administration time, administration frequency, and administration plan may be adjusted according to situations. In this example, mouse melanoma tumor tissues and cancer cells are lysed with 8M urea and dissolved. Then, the lysis components of tumor tissues and cancer cells are loaded into a nanoparticle system in a mass ratio of 1:1, and dendritic cells are activated by the particle system to prevent cancer. In this example, nanoparticles loaded with four peptide neoantigens, B16-M20 (Tubb3, FRRKAFLHWYTGEAMDEMEFTEAESNM), B16-M24 (Dag1, TAVITPPTTTTKKARVSTPKPATPSTD), B16-M46 (Actn4, NHSGLVTFQAFIDVMSRETTDTDTADQ) and TRP2: 180-188 (SVYDFFVWL), are used as control nanoparticles to analyze the effects of nanoparticles loaded with whole cell antigens and nanoparticles loaded with multiple peptide neoantigens in the preparation of dendritic cell vaccine. In this example, after loading whole cell antigens inside and on the surface of nanoparticles, biocalcified the nanoparticles and then co-incubated with dendritic cells.

### (1) Lysis of Tumor Tissues and Cancer Cells

Collected mouse B16F10 melanoma tumor tissues and cultured cancer cells, then used 8M urea to lyse and dissolve whole cell components of tumor tissues and cancer cells and then mixed the tumor tissue components and cancer cell components in a mass ratio of 1:4.

### (2) Preparation of the Nanoparticle System

In this example, nanoparticle system and blank nanoparticles used as controls are prepared by double emulsion method in solvent evaporation. The molecular weight of nanoparticle preparation materials PLGA is 7 KDa-17 KDa, and immune adjuvants used are CpG and Poly(I:C) and are distributed inside the nanoparticles. The preparation method is as follows. During the preparation process, the antigens are loaded inside the nanoparticles by double emulsion method. After loading the antigens (lysis components) inside, resuspend the 100 mg PLGA nanoparticles with 18 mL PBS. Then adding 9 mL of tumor tissue and cancer cell lysate (60 mg/mL) dissolved in 8M urea. Collecting the precipitate after centrifugation at 10000 g for 20 minutes after room temperature reaction for 10 minutes. Then resuspending the 100 mg PLGA nanoparticles in 20 mL DMEM medium, adding 200 mL μL of CaCl₂) (1 mM), and reacted at 37° C. for two hours. Collecting the precipitate after centrifugation at 10000 g for 20 minutes, and centrifugal washing twice after resuspending with ultrapure water. Resuspending the 100 mg nanoparticles in 10 mL RPMI 1640 medium and co-incubated them with dendritic cells. The average particle size of the nanoparticles is about 320 nm. Approximately 150 μg of protein or peptide components are loaded onto 1 mg of PLGA nanoparticles. The preparation method of control nanoparticles loaded with multiple antigen peptides is the same as above. The size of control nanoparticles is about 310 nm, and about 150 μg of antigen peptides are loaded onto 1 mg of PLGA nanoparticles.

### (3) Preparation of Dendritic Cells

Same as Example 1.

### (4) Activation of Dendritic Cells

Same as Example 1.

### (5) Dendritic Cell Cancer Vaccine for Preventing Cancer

Selecting 6-8 weeks-old female C57BL/6 as model mice to prepare tumor-bearing mice. Vaccine group subcutaneously injected 100 μL dendritic cells (1,000,000) respectively on the 35th, 28th, 21st, 14th, and 7th day before tumor inoculation. PBS control group subcutaneously injected 100 μL PBS respectively on the 35th, 28th, 21st, 14th, and 7th day before tumor inoculation. The control vaccine group subcutaneously injected 100 μL dendritic cells (1,000,000) stimulated by control nanoparticles loaded with peptide antigens respectively on the 35th, 28th, 21st, 14th, and 7th day before tumor inoculation. On day 0, 1.5×10⁵ B16F10 melanoma cells are subcutaneously injected into each mouse. The volume of mouse tumors is recorded every three days starting from the third day. The volume of tumors is calculated by formula v=0.52×a×b², wherein v is the volume of tumor, a is the length of tumor, and b is the width of tumor. The mouse is considered dead and euthanized the mouse when the volume of mouse tumors exceeded 2000 mm³ in mouse survival test.

### (6) Experimental Result

As shown in FIG. 13, compared with control groups, the tumor growth rates of mice in the vaccine groups are significantly slower, and tumors in some mice disappeared and healed. Moreover, dendritic cell vaccine activated by nanoparticles loaded with whole cell components are more effective than dendritic cell vaccine activated by nanoparticles loaded with several antigen peptides. This indicates that the dendritic cell vaccine activated in vitro by nanoparticles loaded with whole cell components as described in the present disclosure can effectively prevent cancer.

Obviously, the above examples are only examples provided for clear explanation instead of the limitations on the embodiments. For those skilled in the art, different forms of changes or variations can be made based on the above explanation. It is unnecessary and impossible to exhaustively list all embodiments here. The obvious changes or variations arising herein are still within the claim of the present disclosure.

1. A dendritic cell cancer vaccine, wherein the dendritic cell cancer vaccine is obtained by activating dendritic cells in vitro with delivery particles loaded with cell components, wherein
the delivery particles are nanoparticles and/or micronparticles,
the cell components are derived from water-soluble components and/or non-water-soluble components of cancer cells and/or tumor tissues, and
the activating is co-incubating the delivery particles loaded with the cell components with the dendritic cells.

2. The dendritic cell cancer vaccine according to claim 1, wherein the dendritic cells are autologous dendritic cells and/or allogeneic dendritic cells.

3. The dendritic cell cancer vaccine according to claim 1, wherein a method for loading the cell components onto surfaces of the delivery particles comprising at least one of adsorption, covalent bonding, charge interaction, hydrophobic interaction, one-step or multi-step solidification, mineralization, and encapsulation.

4. The dendritic cell cancer vaccine according to claim 1, wherein the surfaces of the delivery particles is loaded with one or more layers of the cell components, wherein, there are modifiers between the layers when loaded with a plurality of the layers of the cell components.

5. The dendritic cell cancer vaccine according to claim 1, wherein the non-water-soluble components are loaded to the delivery particles after solubilization, and a solubilizer used is selected from at least one of urea, guanidine hydrochloride, sodium deoxycholate, SDS, glycerol, alkaline solution with pH greater than 7, acidic solution with pH less than 7, protein degrading enzyme, albumin, lecithin, inorganic salt, Triton, Tween, DMSO, acetonitrile, ethanol, methanol, DMF, propanol, isopropanol, acetic acid, cholesterol, amino acid, glycoside, choline, Brij™-35, octaethylene glycol monododecyl ether, CHAPS, Digitonin, lauryldimethylamine oxide, IGEPAL® CA-630, dichloromethane, and ethyl acetate.

6. The dendritic cell cancer vaccine according to claim 1, wherein immunopotentiating adjuvants are loaded inside the delivery particles, and/or
the immunopotentiating adjuvants are loaded on the surfaces of the delivery particles.

7. The dendritic cell cancer vaccine according to claim 1, wherein the surfaces of the delivery particles are connected with target heads that actively target the dendritic cells.

8. The dendritic cell cancer vaccine according to claim 1, wherein the water-soluble components and/or the non-water-soluble components are loaded inside the delivery particles, and/or
the water-soluble components and/or the non-water-soluble components are loaded on the surfaces of the delivery particles.

9. Application of the dendritic cell cancer vaccine according to claim 1 in preparing drugs used for treating or preventing a cancer.

10. The application according to claim 9, wherein multiple-dose administration before the onset of the cancer, after the onset of the cancer, or after surgical removal of the tumor tissues to activate an immune system of organisms.
